(19) United States
(12) Patent Application Publication
(10) Pub. No.: US 2025/0090580 A1
(43) Pub. Date: Mar. 20, 2025

(54) METHODS AND COMPOSITIONS FOR USING ACTIVATED LYMPHOCYTES IN THE TREATMENT OF DISEASE

(71) Applicant: Immunolux International Corp, Highland, CA (US)

(72) Inventors: Aladar A. Szalay, Highland, CA (US); Ivan Petrov, Würzburg (DE); Ignazio Caruana, Würzburg (DE); Elena Ekrami, Würzburg (DE)

(21) Appl. No.: 18/293,257

(22) PCT Filed: Jul. 29, 2022
(86) PCT No.: PCT/US2022/038917

§ 371 Date: Jan. 29, 2024

Related U.S. Application Data
(60) Provisional application No. 63/227,991, filed on Jul. 30, 2021.
(60) Provisional application No. 63/320,129, filed on Mar. 15, 2022.

(51) Int. Cl. A61K 35/17; A61K 35/768; A61K 39/00; C12N 5/0783; C12N 7/00

(52) U.S. Cl. CPC A61K 35/17; A61K 35/768; A61K 40/11; A61K 40/31; A61K 40/32; C12N 5/0636; C12N 7/00; C12N 2510/00; C12N 2710/24132

(57) ABSTRACT

Described herein are methods and compositions for treating a disease, e.g. cancer (including solid tumors) inflammatory disease, autoimmune disease, or fibrosis. Method for treating a disease in a subject, the method including administering to the subject a composition containing an oncolytic virus and an immune cell, wherein the immune cell is infected with an oncolytic virus.

### CROSS-REFERENCE TO RELATED APPLICATIONS

This application claims the benefit of U.S. Provisional Application No. 63/227,991, filed Jul. 30, 2021, and U.S. Provisional Application No. 63/320,129, filed Mar. 15, 2022, which are incorporated herein by reference in their entireties and for all purposes.

### BACKGROUND

Cancer is a worldwide health emergency, and at least 1 out of 3 people will be personally effected by a cancer diagnosis over their lifetime. While the incidence of some cancers (lung) is decreasing, mainly due to the declining use of tobacco products, overall the number of cancer cases is on the rise by 30% from 1973 to 2015.

There is a large unmet need for new, paradigm shifting therapies to address undruggable, untreatable, recurrent or treatment-nonresponsive cancers, despite the fact that many treatments currently exist and are used to successfully treat many types of cancer.

New methods of treating refractory cancers are needed.

### BRIEF SUMMARY OF THE INVENTION

Described herein are compositions and methods for treating a disease in a subject in need thereof by administering to the subject an oncolytic virus and an immune cell. In embodiments, the disease is cancer. In embodiments, the disease is an infection, autoimmune disease, fibrosis, or inflammatory disease.

In an aspect, provided herein, is a composition containing an oncolytic virus and an immune cell, wherein the immune cell is infected by the oncolytic virus.

In an aspect, provided herein, is a composition containing an oncolytic virus and a T or NK cell expressing a chimeric antigen receptor (CAR) (CAR-T cell or CAR-NK cell). In embodiments, the CAR is a bi-specific CAR, inducible CAR, or universal CAR.

In an aspect, provided herein, is a composition containing an oncolytic virus and a T cell expressing an exogenous T cell receptor (TCR).

In an aspect, provided herein, is a pharmaceutical composition containing an oncolytic virus and an immune cell, wherein the immune cell is infected by the oncolytic virus, and a pharmaceutically acceptable excipient.

In an aspect, provided herein, is a method for treating a disease in a subject, the method including administering to the subject a composition containing an oncolytic virus and an immune cell, wherein the immune cell is infected with an oncolytic virus.

In an aspect, provided herein, is a method for treating cancer in a subject in need thereof, the method comprising administering to the subject a composition containing an oncolytic virus and an immune cell, wherein the immune cell is infected with an oncolytic virus.

In an aspect, provided herein, is a method for making a composition containing an oncolytic virus and lymphocytes. In embodiments, the method includes obtaining lymphocytes from a subject, and contacting the lymphocytes with an oncolytic virus to form oncolytic virus infected lymphocytes.

### BRIEF DESCRIPTION OF THE FIGURES

FIG. 1. Shows chimeric antigen receptor (CAR) redirected T cells (CAR T cells) before (left panel) and 48 hours (h) after infection with vaccinia virus (LIVP1.1.1).

FIG. 2. Shows the ability of vaccinia to infect non-transduced T cells (not expressing a CAR; “NT”) compared to CAR T cells. The left plot shows the total virus infected area (μm²/image) over time (hours), based on expression of TurboFP365, for three sets of CAR T cells vs. NT T cells generated from three patients. The right plot shows the total virus infected area (μm²/image) at the time course end point (65 hours) for each condition. Numbers indicate patient from which T cells were generated.

FIG. 3. Shows total plaque forming units (PFU) from samples taken from conditioned media. In the top two plots, Lister strain (variants, expressing TurboFP635 or ig-GFP, respectively) was used, and in the bottom plot, Copenhagen strain (TurboFP635-expressing) was used. In each case, approximately 7×10⁵ cells were infected at approximately 1.7 MOI and washed after 2 hours. The total plaque forming units (PFU) were counted and plotted at 24, 48, 72, and 96 hours after infection.

FIGS. 4A-4Q are fluorescent micrographs showing the progression of infection and cell death of SH-SY5Y human neuroblastoma cells over time after co-culture with vaccinia-infected CAR T cells. The SH-SY5Y cells were co-cultured 2:1 (SH-SY5Y cells:CAR T cells) with CAR T cells previously infected with vaccinia virus. Virus-expressing cells are red due to TurboFP635 expression. SH-SY5Y cells are green (i.e., expressing GFP) prior to infection; yellow indicates co-expression of virus and GFP. FIG. 4A: 1 h after co-culture. FIG. 4B: 5 h after co-culture. FIG. 4C: 10 h after co-culture. FIG. 4D: 15 h after co-culture. FIG. 4E: 20 h after co-culture. FIG. 4F: 1 day (d) 1 h after co-culture. FIG. 4G: 1 d 5 h after co-culture. FIG. 4H: 1 d 10 h after co-culture. FIG. 4I: 1 d 15 h after co-culture. FIG. 4J: 1 d 20 h after co-culture. FIG. 4K: 2 d 1 h after co-culture. FIG. 4L: 2 d 6 h after co-culture. FIG. 4M: 2 d 11 h after co-culture. FIG. 4N: 2 d 16 h after co-culture. FIG. 4O: 2 d 21 h after co-culture. FIG. 4P: 3 d 1 h after co-culture. FIG. 4Q: 3 d 3 h after co-culture.

FIG. 5. Shows the ability of uninfected CAR T cells (top row) compared to CAR T cells infected with vaccinia virus (bottom row) to kill SH-SY5Y neuroblastoma cells. SH-SY5Y cells were co-cultured 2:1 (SH-SY5Y cells:CAR T cells) with the indicated CAR T cells, and a fluorescent micrograph taken at 0 h (left) and 75 h (right) after co-culture. There was a significant enhancement of tumor cell eradication over 75 hour co-culture for the tumor cells co-cultured with the vaccinia virus-infected CAR T cells as compared to the tumor cells co-cultured with CAR T cells alone. SH-SY5Y cells are green (cells express GFP); vaccinia-infected cells are red.

FIG. 6. Shows a plot of the of the total green object area (viable SH-SY5Y cells) over time (hours), indicating the amount of intact tumor cells in culture under the following conditions: SH-SY5Y alone, SH-SY5Y with vaccinia virus (0.01 MOI), SH-SY5Y with CAR T cells (2:1 co-culture), or SH-SY5Y with vaccinia virus-infected CAR T cells (2:1 co-culture). A fluorescent micrograph (e.g., as in FIG. 5) was taken at the indicated time point for determination of total green object area.

FIG. 7. Shows the number of vaccinia-infected T cells under the indicated conditions. The two plots on the left represent FACS analysis data (%) of Vaccinia-infected, polyclonally activated T cells; the two plots on the right show FACS analysis of modulation of CAR expression after Vaccinia virus infection. Right plot shows CAR expression under each condition, as % CAR positive cells. Vaccinia virus infection is more homogenous in CAR redirected T cells compared to non-gene modified T cells, and vaccinia infection does not negatively affect the level of CAR T cell expression. No infected—NT: Uninfected T cells; No infected—CAR: Uninfected CAR T cells; Infected—NT: Vaccinia-infected T cells; Infected—CAR: Vaccinia-infected CAR T cells.

FIG. 8. The left plot shows that vaccinia virus infects T cells, NK cells, and NK-T cells. FACS analysis of subpopulation of cells present in patient derived CAR gene-modified T cells. The left panel shows percentages of T, NK, NK-T populations in the non-gene modified (NT-non-transduced) T cells, and gene-modified CAR after Vaccinia infection. On the right, the graph shows FACS analysis of CD8 and CD4 compartments in CD3+ population with or without Vaccinia infection in both NT and CAR+ patient derived lymphocytes. CAR detection: anti CAR idiotype. T cell populations include: CD45/CD3, NK-T: CD45/CD3/CD56, NK: CD45/CD56+/CD3−. The right plot shows that vaccinia virus infects CD4 and CD8 subpopulations of T cells. The percentage of infection in CD8 cells is greater than in CD4 cells in this example. The right panel shows % positive cells vs., from the left to the right: VV+: only VV positive cells, CAR+: only CAR positive cells, +/+: cells positive for both VV and CAR, −/−: cells that are double negative for VV and CAR.

FIG. 9A. The vaccinia infection induces a maturation of T cells mainly in the CD8 compartment, with a minor amount of naïve cells and major amount of effector cells. Shown here are two plots of % of positively VV infected cells vs. cell type. Cell types represented are naïve=naïve T cells, CM=central memory T cells, EM=effector memory T cells, and ET terminal effector memory T cells, and NT=non-transduced. The left bar graph shows a comparison of memory NT T cells (CD4+/CD8+) and memory CAR T cells (CD4+/CD8+) infected by VV. The right bar graph shows memory CAR T cells (CD4+/CD8+) infected by VV.

FIG. 9B. The vaccinia infection induces a maturation of T cells mainly in the CD8 compartment, with a minor amount of naïve cells and major amount of effector cells. Shown here are two plots of % of positively VV infected cells vs. cell type. Cell types represented are naïve=naïve T cells, CM=central memory T cells, EM=effector memory T cells, and ET terminal effector memory T cells, and NT=non-transduced. The left bar graph shows a comparison of maturation of memory CAR T cells (CD4+/CD8+) not infected or infected by VV. The right bar graph shows a comparison of maturation of CD4/CD8−/−NT or CAR T cells not infected or infected by VV.

### DETAILED DESCRIPTION

After reading this description it will become apparent to one skilled in the art how to implement the invention in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description of the invention is divided into various sections only for the reader's convenience and disclosure found in any section may be combined with that in another section. Titles or subtitles may be used in the specification for the convenience of a reader, which are not intended to influence the scope of the present invention.

### I. Definitions

Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method in steps. Embodiments defined by each of these transition terms are within the scope of this invention.

The terms “disease” or “condition” refer to a state of being or health status of a patient or subject capable of being treated with the compounds or methods provided herein. The disease may be an autoimmune disease. The disease may be an inflammatory disease. The disease may be an infectious disease.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence (or recurrence) of disease symptoms in a patient. As indicated above, the prevention may be complete (no detectable symptoms) or partial, such that fewer symptoms are observed than would likely occur absent treatment.

“Patient,” “subject,” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, pigs, deer, and other non-mammalian animals. In some embodiments, a patient is human. In embodiments, the human is a pediatric patient. In embodiments, a patient is a domesticated animal (e.g., goat, sheep, cow, horse, etc.). In embodiments, a patient is a companion animal, including but not limited to canine, feline, rodent (mouse, rat, gerbil, hamster, guinea pig, chinchilla, and the like), rabbit, ferret, etc.

An “effective amount” is an amount sufficient for a compound to accomplish a stated purpose relative to the absence of the compound (e.g. achieve the effect for which it is administered, treat a disease, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As is well known in the art, therapeutically effective amounts for use in humans can also be determined from animal models. For example, a dose for humans can be formulated to achieve a dose that has been found to be effective in animals. The dosage in humans can be adjusted by monitoring effectiveness and adjusting the dosage upwards or downwards, as described herein. Adjusting the dose to achieve maximal efficacy in humans based on the methods described herein and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the composition being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. In some embodiments, treatment is initiated with smaller dosages which are less than the optimum dose of the composition. Thereafter, the dosage is increased by small increments until the optimum effect under circumstances is reached. Dosage amounts and intervals can be adjusted individually to provide levels of the administered composition effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intra-cerebro-ventricular, intrapleural, intra-parencymal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, etc. Administration also includes direct administration, e.g., directly to a site of inflammation. Direct administration may be via guided delivery, e.g., magnetic resonance imaging (MRI)-guided delivery. In embodiments, the administering does not include administration of any active agent other than the recited active agent.

“Co-administer” is meant that a composition described herein is administered at the same time, just prior to, or just after the administration of one or more additional therapies. The compositions provided herein can be administered alone or can be co-administered to the patient. Co-administration is meant to include simultaneous or sequential administration of the compositions individually or in combination (more than one composition). Thus, the preparations can also be combined, when desired, with other active substances.

The term “autologous,” “autologous cell” or “autologous transplantation” as used herein in relation to cell transplantation indicates that the donor and recipient of the cells is the same individual. The term “allogenic,” “allogenic cell” or “allogenic transplantation” as used herein in relation to cell transplantation indicates that the donor and recipient of the cells are different individuals of the same species.

The term “lymphocytes” as used herein refers to immune cells made in the bone marrow and found in the blood and in lymph tissue. Lymphocytes are a type of white blood cell. Other “immune cells” include, but are not limited to, neutrophils, eosinophils, basophils, mast cells, monocytes, macrophages, dendritic cells, natural killer cells, and lymphocytes.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. In embodiments, the T cells are α/β T cells (express TCRα/β). In embodiments, the T cells are γ/δ T cells (express TCRγ/δ). Different types of T cells can be distinguished by use of T cell detection agents.

A “memory T cell” is a T cell that has previously encountered and responded to its cognate antigen during prior infection, encounter with cancer or previous vaccination. At a second encounter with its cognate antigen memory T cells can reproduce (divide) to mount a faster and stronger immune response than the first time the immune system responded to the pathogen.

A “regulatory T cell” or “suppressor T cell” is a lymphocyte which modulates the immune system, maintains tolerance to self-antigens, and prevents autoimmune disease.

As used herein, “CAR-T” or “chimeric antigen receptor T cell” refers to a T cell that has been genetically engineered to contain an artificial T cell receptor.

A “TCR” or “T cell receptor” refers to a protein complex found on the surface of T cells or T lymphocytes.

An “oncolytic virus” is a virus that can infect and kill cancer cells. Examples of oncolytic viruses include, but are not limited to, adenoviruses, herpes viruses, measles viruses, coxsackie viruses, polioviruses, reoviruses, poxviruses, vaccinia viruses, Vesicular stomatitits virus, senecavirus, RIGVIR, semliki forrest virus, maraba virus and Newcastle disease viruses.

The term “Cluster of Differentiation protein” or “CD protein” or “cluster of designation” or “classification determinant” as used herein includes any of the cell surface proteins identified as targets for immunophenotyping cells. CD proteins can act as receptors or ligands for participation in immune responses. CD proteins include, but are not limited to, CD3, CD4, CD5, CD7, CD8, CD19, CD20, CD22, CD30, CD33, CD44v6, CD123, CD138, CD171, CD4, CD8 or CD45RA+.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

The term “infection” or “infectious disease” refers to a disease or condition that can be caused by organisms such as a bacterium, virus, fungi or any other pathogenic microbial agents. In embodiments, the infectious disease is caused by a pathogenic bacteria. Pathogenic bacteria are bacteria which cause diseases (e.g., in humans). In embodiments, the infectious disease is a bacteria associated disease (e.g., tuberculosis, which is caused by Mycobacterium tuberculosis). Non-limiting bacteria associated diseases include pneumonia, which may be caused by bacteria such as Streptococcus and Pseudomonas; or foodborne illnesses, which can be caused by bacteria such as Shigella, Campylobacter, and Salmonella. Bacteria associated diseases also includes tetanus, typhoid fever, diphtheria, syphilis, and leprosy. In embodiments, the disease is Bacterial vaginosis (i.e. bacteria that change the vaginal microbiota caused by an overgrowth of bacteria that crowd out the Lactobacilli species that maintain healthy vaginal microbial populations) (e.g., yeast infection, or Trichomonas vaginalis); Bacterial meningitis (i.e. a bacterial inflammation of the meninges); Bacterial pneumonia (i.e. a bacterial infection of the lungs); Urinary tract infection; Bacterial gastroenteritis; or Bacterial skin infections (e.g. impetigo, or cellulitis). In embodiments, the infectious disease is a Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, or Vibrio cholera infection.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refers to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs. The term “tumor” or “tumor cell,” as used herein, refers to any type of tumor, including solid tumors or non-solid tumors, dispersed tumors, metastatic or disseminated tumors, or tumor cells from any form of tumor.

The term “fibrosis” refers to any process of pathological wound healing or development of fibrous connective tissue in response to damage or injury. Types of fibrosis include, but are not limited to, pulmonary fibrosis (replacement fibrosis, focal fibrosis, diffuse parencymal lung disease, cystic fibrosis, fibrothorax, idiopathic pulmonary fibrosis, and radiation-induced lung injury), liver fibrosis (bridging fibrosis, cirrhosis, senescence of hepatic stellate cells), kidney fibrosis (CYR61 induction of cellular senescence), glial scars, heart fibrosis (interstitial fibrosis, replacement fibrosis, and myocardial fibrosis) arterial stiffness, athrofibrosis, chronic kidney disease, Chrone's disease, Dupuytren's contracture, keloid, mediastinal bibrosis, myelofibrosis, Peyronie's disease, nephrogenic systemic fibrosis, progressive massive fibrosis, retroperitoneal bibrosis, scleroderma/systemic sclerosis, and adhesive capsulitis.

The term “fibroblast” refers to a type of cell that secretes extracellular matrix (ECM) components, including, but not limited to, collagen, glycoaminoglycans, reticular fibers, elastic fibers, and ground substance.

The term “vaccine” as used herein, refers to any type of biological preparation contributing to or soliciting active immune responses against a particular disease or pathogen. Such biological preparation can include, but is not limited to, an antigen derived from a disease-causing agent or a portion of an antigen derived from a disease-causing agent. Such biological preparation can also be in the form of live attenuated preparation, including live, or weakened or modified disease causing agents or pathogens; or in the form of inactivated or killed disease-causing agents or pathogens. Alternative forms of such biological preparation further include, but are not limited to, the forms of subunit, toxoid, conjugate, DNA and recombinant vectors, or any suitable forms that might become developed or available in the future for soliciting active immune responses there against.

It should be noted that in some embodiments, while the term “vaccine” is used herein, the vaccine need not provide significant immunity against smallpox (or any other pathogen), so long as it is effective against a disease as described herein. For example, the vaccine may be any immunogenic or infectious composition that treats the disease. In some cases, the term is used to identify certain materials or compositions, and not necessarily the ability of material or composition to provide immunity against smallpox, for example. The virus can be from any strain of virus, including for example, one or more listed below and elsewhere herein, including those that are not part of approved or contemplated vaccines.

As used herein, “virus” refers to any of a large group of entities referred to as viruses. Viruses typically contain a protein coat surrounding an RNA or DNA core of genetic material, but no semipermeable membrane, and are capable of growth and multiplication only in living cells. Viruses for use in the methods provided herein include, but are not limited, to a poxvirus, adenovirus, herpes simplex virus, Newcastle disease virus, vesicular stomatitis virus, mumps virus, influenza virus, measles virus, reovirus, human immunodeficiency virus (HIV), hanta virus, myxoma virus, cytomegalovirus (CMV), lentivirus, and any plant or insect virus.

As used herein, “heterologous nucleic acid” refers to a nucleic acid, DNA or RNA, which has been introduced into a virus or a cell (or the cell's ancestor). Such heterologous nucleic acid may comprise the sequence and operable regulatory elements for genes. For example, the heterologous nucleic acid may comprise a selection marker gene, a suicide gene, or a gene expressing a useful protein product that is not expressed endogenously, or expressed endogenously at low levels.

As used herein, the term “concurrently” as referring to administration of an oncolytic virus and a cell, refers to administration within 48 hours of each other. In some embodiments, the oncolytic virus and cell are administered within 36 hours of each other, within 24 hours of each other, within 12 hours of each other, within 10 hours of each other, within 8 hours of each other, within 6 hours of each other, within 4 hours of each other, within two hours of each other, within 1 hour of each other.

The term “autologous,” “autologous cell” or “autologous transplantation” as used herein in relation to cell transplantation indicates that the donor and recipient of the cells is the same individual. The term “allogenic,” “allogenic cell” or “allogenic transplantation” as used herein in relation to cell transplantation indicates that the donor and recipient of the cells are different individuals of the same species.

“Pharmaceutically acceptable excipient” and “pharmaceutically acceptable carrier” refer to a substance that aids the administration of an active agent to and/or absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

### II. Oncolytic Viruses

Oncolytic viruses are viruses that target cancer cells. Oncolytic viruses can be genetically engineered to selectively replicate in and lyse cancer cells, while avoiding healthy cells. Oncolytic viruses destroy cancer cells by either: 1) directly infecting a cancer cell, and lysing it, or 2) recruiting and directing a refreshed host immune response to the cancer cells.

The potential for oncolytic viruses as a treatment for cancer was initially discovered after a patient with myelogenous leukemia was infected with influenza, and went into spontaneous remission. Viruses from many classes have been genetically engineered to be oncolytic, including, but are not limited to: adenoviruses, herpes viruses, measles viruses, coxsackie viruses, polioviruses, reoviruses, poxviruses, vaccinia viruses, Vesicular stomatitits virus, senecavirus, RIGVIR, semliki forrest virus, maraba virus and Newcastle disease viruses. Viral components are the targets for genetic engineering, and include: adding tumor-specific promoters, viral gene knockouts, viral capsid modification, and expression of immune system-activating agents including antibodies, cytokines, and costimulatory molecules.

Variola virus is the cause of smallpox. Unlike variola virus, vaccinia virus does not normally cause systemic disease in immune-competent individuals and it has therefore been used as a live vaccine to immunize against smallpox. Smallpox has been eradicated as a natural disease due to successful worldwide vaccination with vaccinia virus. Routine smallpox vaccination has been discontinued for many years, except for people at higher risk of poxvirus infections (e.g., laboratory workers). Although the United States discontinued routine childhood immunization against smallpox in 1972, the use of smallpox vaccine is generally considered safe for pediatric use.

Attenuated strains derived from a pathogenic virus can be used for the manufacturing of a live vaccine. Non-limiting examples of viral strains that have been used as a smallpox vaccine include but are not limited to the Lister (also known as Elstree), New York City Board of Health (“NYCBH strain”), Dairen, Ikeda, LC16M8, Western Reserve (WR), Copenhagen, Tashkent, Tian Tan, Wyeth, IHD-J, and IHD-W, Brighton, Ankara, MVA, Dairen I, LIPV, LC16MO, LIVP, WR 65-16, EM63, and Connaught strains. In some embodiments, the smallpox vaccine as disclosed herein is an attenuated New York City Board of Health (NYCBOH) strain of vaccinia virus. In some embodiments, the NYCBOH strain of vaccinia virus may be ATCC VR-118 or CJ-MVB-SPX.

Surprisingly, an oncolytic virus can infect immune cells, and the infected immune cells can be used to target and kill tumor cells. In embodiments, the oncolytic virus is a poxvirus.

In embodiments, the poxvirus is vaccinia virus. In some embodiments, the vaccinia virus is selected from Dryvax, ACAM1000, ACAM2000, Lister, EM63, LIVP, Tian Tan, Copenhagen, Western Reserve, Modified Vaccinia Ankara (MVA), New York City Board of Health, Dairen, Ikeda, LC16M8, Tashkent, Wyeth, IHD-J, IHD-W, Brighton, Dairen I and Connaught strains. In some embodiments, the vaccinia virus strain is Drayvax. In some embodiments, the vaccinia virus strain is ACAM1000. In some embodiments, the vaccinia virus strain is ACAM2000. In some embodiments, the vaccinia virus strain is Lister. In some embodiments, the vaccinia virus strain is EM63. In some embodiments, the vaccinia virus strain is LIVP. In some embodiments, the vaccinia virus strain is Tian Tan. In some embodiments, the vaccinia virus strain is Copenhagen. In some embodiments, the vaccinia virus strain is Western Reserve. In some embodiments, the vaccinia virus strain is Modified Vaccinia Ankara (MVA). In some embodiments, the vaccinia virus strain is Ikeda. In some embodiments, the vaccinia virus strain is New York City Board of Health. In some embodiments, the vaccinia virus strain is Dairen. In some embodiments, the vaccinia virus strain is LC16M8. In some embodiments, the vaccinia virus strain is Tashkent. In some embodiments, the vaccinia virus strain is Wyeth. In some embodiments, the vaccinia virus strain is IHD-J. In some embodiments, the vaccinia virus strain is IHD-W. In some embodiments, the vaccinia virus strain is Brighton. In some embodiments, the vaccinia virus strain is Dairen I. In some embodiments, the vaccinia virus strain is Connaught.

In embodiments, the poxvirus is oncolytic. In some embodiments, the vaccinia virus is oncolytic. In some embodiments, the oncolytic vaccinia virus selected from Dryvax, ACAM1000, ACAM2000, Lister, EM63, LIVP, Tian Tan, Copenhagen, Western Reserve, Modified Vaccinia Ankara (MVA), New York City Board of Health, Dairen, Ikeda, LC16M8, Tashkent, Wyeth, IHD-J, IHD-W, Brighton, Dairen I and Connaught strains. In some embodiments, vaccinia virus strain Dryvax is oncolytic. In some embodiments, vaccinia virus strain Dryvax is ACAM1000. In some embodiments, vaccinia virus strain ACAM2000 is oncolytic. In some embodiments, vaccinia virus strain Lister is oncolytic. In some embodiments, vaccinia virus strain EM63 is oncolytic. In some embodiments, vaccinia virus strain LIVP is oncolytic. In some embodiments, vaccinia virus strain Tian Tan is oncolytic. In some embodiments, vaccinia virus strain Copenhagen is oncolytic. In some embodiments, vaccinia virus strain Western Reserve is oncolytic. In some embodiments, vaccinia virus strain Modified Vaccinia Ankara (MVA) is oncolytic. In some embodiments, vaccinia virus strain New York City Board of Health is oncolytic. In some embodiments, vaccinia virus strain Dairen is oncolytic. In some embodiments, vaccinia virus strain Ikeda is oncolytic. In some embodiments, vaccinia virus strain LC16M8 is oncolytic. In some embodiments, vaccinia virus strain Tashkent is oncolytic. In some embodiments, vaccinia virus strain Wyeth is oncolytic. In some embodiments, vaccinia virus strain IHD-J is oncolytic. In some embodiments, vaccinia virus strain IHD-W is oncolytic. In some embodiments, vaccinia virus strain Brighton is oncolytic. In some embodiments, vaccinia virus strain Dairen I is oncolytic. In some embodiments, vaccinia virus strain Connaught is oncolytic.

In embodiments, the oncolytic virus does not lyse an immune cell. In some embodiments, the oncolytic virus does not lyse an allogenic immune cell. In some embodiments, the oncolytic virus does not lyse an autologous immune cell. In some embodiments, the oncolytic virus does not lyse a lymphocyte. In embodiments, “does not lyse” indicates that the virus does not lyse the cell for at least 12 hours, 24 hours, 36 hours, 48 hours, 60 hours, 72 hours, 86 hours, 98 hours or more after infection of the cell with the virus.

U.S. Pat. No. 10,105,436, which is incorporated herein by reference in its entirety for all that is taught therein, describes poxviruses, including smallpox vaccines, that can be used in the methods and compositions described herein.

### III. Immune Cells

Any method for making and using CARs, CAR T cells, or CAR-NK cells may be used with the compositions and methods described herein. Methods of making and using CAR-T cells are well known in the art, for example as disclosed in U.S. Pat. No. 9,328,156, which is incorporated herein by reference in its entirety for all that is taught therein.

In an aspect, provided herein, is a composition containing an oncolytic virus and an immune cell, wherein the immune cell is infected by the oncolytic virus.

In embodiments, the immune cell is a lymphocyte. In some embodiments, the lymphocyte is selected from: a T cell, an α/β T cell, a γ/δ T cell, a B cell, an NK cell, an NK-T cell, a myeloid cell, or a lymphoid dendritic cell. In some embodiments, the lymphocyte is an α/β T cell. In some embodiments, the lymphocyte is a γ/δ T cell. In some embodiments, the lymphocyte is a B cell. In some embodiments, the lymphocyte is a NK cell. In some embodiments, the lymphocyte is a NK-T cell. In some embodiments, the lymphocyte is a myeloid cell. In some embodiments, the lymphocyte is a lymphoid dendritic cell.

In embodiments, the immune cell is derived from a subject to be treated with the composition (autologous). In embodiments, the immune cell is allogeneic to a subject to be treated with the composition.

In embodiments, the immune cell is genetically modified. In some embodiments, the immune cell is genetically modified to target a target cell. In some embodiments, the target cell is a cancer cell, a pathogen, or a pathogen-infected cell. In some embodiments, the target cell is a cancer cell. In some embodiments, the target cell is a pathogen. In some embodiments, the target cell is a pathogen-infected cell. In some embodiments, the target cell is an autoimmune cell.

In an aspect, provided herein, is a composition containing an oncolytic virus and an immune cell expressing a chimeric antigen receptor (CAR). In embodiments, the immune cell is a lymphocyte. In an aspect, provided herein, is a composition containing an oncolytic virus and a T cell or NK cell expressing a chimeric antigen receptor (CAR) (CAR-T cell or CAR-NK cell). In embodiments, the CAR is a bi-specific CAR, inducible CAR, or universal CAR.

In an aspect, provided herein, is a composition containing an oncolytic virus and a T cell expressing an exogenous T cell receptor (TCR).

In some embodiments, the T cell is autologous. In some embodiments, the T cell is allogeneic.

In embodiments, a chimeric antigen receptor (CAR)-T cell is also administered to the subject. In embodiments, the CAR targets an antigen associated with the disease. In some embodiments, the CAR-T cell is autologous. In some embodiments, the CAR-T cell is allogeneic.

In embodiments, a chimeric antigen receptor (CAR)-T cell is also administered to the subject. In embodiments, the CAR targets an antigen associated with the disease. In embodiments, the CAR-T cell is autologous. In embodiments, the CAR-T cell is allogeneic.

In an aspect, provided herein, is a composition containing an oncolytic virus and an immune cell, wherein the immune cell is infected by the oncolytic virus.

In embodiments, the oncolytic virus is a poxvirus. In embodiments, the oncolytic virus is vaccinia virus. In some embodiments, the oncolytic vaccinia virus is selected from Dryvax, ACAM1000, ACAM2000, Lister, EM63, LIVP, Tian Tan, Copenhagen, Western Reserve, Modified Vaccinia Ankara (MVA), New York City Board of Health, Dairen, Ikeda, LC16M8, Tashkent, Wyeth, IHD-J, IHD-W, Brighton, Dairen I and Connaught strains.

In embodiments, the oncolytic virus does not lyse an immune cell. In embodiments, the virus does not lyse the immune cell for at least 1 day to at least 10 days after infection. In embodiments, the virus does not lyse the immune cell for at least 1 day to at least 5 days after infection. In embodiments, the virus does not lyse the immune cell for at least 1 day after infection. In embodiments, the virus does not lyse the immune cell for at least 2 day after infection. In embodiments, the virus does not lyse the immune cell for at least 3 day after infection. In embodiments, the virus does not lyse the immune cell for at least 4 day after infection. In embodiments, the virus does not lyse the immune cell for at least 5 day after infection. In embodiments, the virus does not lyse the immune cell for at least 6 day after infection. In embodiments, the virus does not lyse the immune cell for at least 7 day after infection. In embodiments, the virus does not lyse the immune cell for at least 8 day after infection. In embodiments, the virus does not lyse the immune cell for at least 9 day after infection. In embodiments, the virus does not lyse the immune cell for at least 10 day after infection. The length of time may be any value or subrange within the recited ranges.

### Methods of Use

### Treatment of Cancer

In embodiments, T cells, e.g. CAR-T cells are administered in combination with the oncolytic virus. In some embodiments, the oncolytic virus infects the T cells prior to administration. In some embodiments, the oncolytic virus infected T cells are administered. Methods for administering T cells, e.g. CAR-T cells, are well known in the art, and can be determined by a skilled clinician.

The methods and compositions disclosed herein can be used to treat any solid tumor or hematologic malignancy. Tumors that can be treated by the methods disclosed herein include, but are not limited to a bladder tumor, breast tumor, prostate tumor, carcinoma, basal cell carcinoma, biliary tract cancer, bladder cancer, bone cancer, brain cancer, CNS cancer, glioma tumor, cervical cancer, choriocarcinoma, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemia, liver cancer, lung cancer, lymphoma, Hodgkin's lymphoma, Non-Hodgkin's lymphoma, melanoma, myeloma, neuroblastoma, oral cavity cancer, ovarian cancer, pancreatic cancer, retinoblastoma, rhabdomyosarcoma, rectal cancer, renal cancer, cancer of the respiratory system, sarcoma, skin cancer, stomach cancer, testicular cancer, thyroid cancer, uterine cancer, and cancer of the urinary system, such as lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, small cell lung cancer, non-small cell lung cancers, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumor, testicular tumor, seminoma, Sertoli cell tumor, hemangiopericytoma, histiocytoma, chloroma, granulocytic sarcoma, corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma, cystadenoma, follicular lymphoma, intestinal lymphosarcoma, fibrosarcoma, and pulmonary squamous cell carcinoma, leukemia, hemangiopericytoma, ocular neoplasia, preputial fibrosarcoma, ulcerative squamous cell carcinoma, preputial carcinoma, connective tissue neoplasia, mastocytoma, hepatocellular carcinoma, lymphoma, pulmonary adenomatosis, pulmonary sarcoma, Rous sarcoma, reticulo-endotheliosis, fibrosarcoma, nephroblastoma, B-cell lymphoma, lymphoid leukosis, retinoblastoma, hepatic neoplasia, lymphosarcoma, plasmacytoid leukemia, swimbladder sarcoma (in fish), caseous lumphadenitis, lung carcinoma, insulinoma, lymphoma, sarcoma, salivary gland tumors, neuroma, pancreatic islet cell tumor, gastric MALT lymphoma and gastric adenocarcinoma.

In some embodiments, the tumor is selected from metastatic melanoma; esophageal and gastric adenocarcinoma; cholangiocarcinoma (any stage); pancreatic adenocarcinoma (any stage); gallbladder cancer (any stage); high-grade mucinous appendix cancer (any stage); high-grade gastrointestinal neuroendocrine cancer (any stage); mesothelioma (any stage); soft tissue sarcoma; prostate cancer; renal cell carcinoma; lung small cell carcinoma; lung non-small cell carcinoma; head and neck squamous cell carcinoma; colorectal cancer; ovarian carcinoma; hepatocellular carcinoma; and glioblastoma.

In some embodiments, the tumor is selected from: glioblastoma, breast carcinoma, lung carcinoma, prostate carcinoma, colon carcinoma, ovarian carcinoma, neuroblastoma, central nervous system tumor, and melanoma.

In some embodiments, the tumor or cancer that can be treated is a childhood or pediatric tumor or cancer. For example, the tumor or cancer can be a leukemia, a lymphoma, a sarcoma, and the like. Non-limiting examples of leukemia include acute lymphoblastic leukemia (ALL) and acute myeloid leukemia (AML). Non-limiting examples of types of lymphomas include Hodgkin disease (or Hodgkin lymphoma) and non-Hodgkin lymphoma (e.g., B and T cell lymphomas). Non-limiting examples of solid tumors or cancers for pediatric patients include brain tumors, Ewing Sarcoma, eye cancer (retinoblastorna), germ cell tumors, Kidney tumors (e.g., Wilms Tumor), liver cancer, neuroblastoma, osteosarcoma, rhabdornyosarcoma, skin cancer (e.g., melanoma), soft tissue sarcoma and thyroid cancer. In some embodiments, the subject is human. In some embodiments, the subject is a pediatric patient. In some embodiments, the subject is a neonate. In some embodiments, the subject is an infant. In some embodiments, the subject is a child. In some embodiments, the subject is an adolescent. In some embodiments, the subject is greater than 12 months in age. In some embodiments the subject is less than 18 years in age.

In embodiments, the method further includes administering to the subject a known treatment for cancer, such as a chemotherapeutic, therapeutic antibody, cancer vaccine, and the like. In embodiments, the oncolytic virus includes a recombinant polynucleotide, wherein the recombinant polynucleotide encodes a therapeutic molecule. In embodiments, the therapeutic molecule treats the cancer.

U.S. Pat. No. 10,105,436, which is incorporated herein by reference in its entirety, describes methods of administering, making, storing, and using compositions comprising poxvirus and cells that can be used in the methods and compositions described herein.

### Treatment of Other Diseases

In an aspect, a method for treating a disease or condition in a subject in need thereof, wherein the disease or condition is not cancer, is provided. The method includes administering to the subject an oncolytic virus and an immune cell. In embodiments, the method further includes treating the disease or condition. In embodiments, treating the disease or condition includes administering to the subject a known treatment for the disease or condition.

In an aspect, a method for treating a disease characterized by chronic inflammation is provided. The method includes administering to the subject an oncolytic virus and an immune cell. In embodiments, the method further includes treating the chronic inflammation. In embodiments, treating the chronic inflammation includes administering to the subject a known treatment for chronic inflammation.

In an aspect, a method for converting chronic inflammation into acute inflammation in a subject in need thereof is provided. The method includes administering to the subject an oncolytic virus and an immune cell.

In an aspect, a method for treating an infection is provided. The method includes administering to the subject an oncolytic virus and an immune cell. In embodiments, the method further includes treating the infection. In embodiments, treating the chronic inflammation includes administering to the subject a known treatment for infection. In embodiments, the infection is infection by a pathogen.

In an aspect, a method for treating an autoimmune disease is provided. The method includes administering to the subject an oncolytic virus and an immune cell. In embodiments, the method further includes treating the autoimmune disease. In embodiments, treating the autoimmune disease includes administering to the subject a known treatment for the autoimmune disease.

In an aspect, a method for treating a fibrosis is provided. The method includes administering to the subject an oncolytic virus and an immune cell. In embodiments, the method further includes treating the fibrosis. In embodiments, treating the fibrosis includes administering to the subject a known treatment for the autoimmune disease.

In embodiments, the oncolytic virus includes a recombinant polynucleotide, wherein the recombinant polynucleotide encodes a therapeutic molecule. In embodiments, the therapeutic molecule treats the disease. In embodiments, the therapeutic molecule is an anti-inflammatory molecule.

In embodiments, the disease is a chronic inflammatory disease. In embodiments, the chronic inflammatory disease is an autoimmune disease. In embodiments, the chronic inflammatory disease is asthma, chronic peptic ulcer, tuberculosis, arthritis, periodontitis, ulcerative colitis, Crohn's disease, sinusitis, active hepatitis, atherosclerosis, dermatitis, inflammatory bowel disease (IBS), systemic lupus, fibromyalgia, Type 1 diabetes, psoriasis, Multiple sclerosis, Addison's disease, Grave's disease, Sjögren's syndrome, Hashimoto's thyroiditis, Myasthenia gravis, vasculitis, pernicious anemia, or celiac disease.

In embodiments, the inflammatory disease is transplant rejection, Dupytren's contracture, peyronies, periodontitis, endometriosis, hepatitis, glomerunephritis, atherscleroisis, cardiovascular disease, arthritis (e.g., osteoarthritis, rheumatoid arthritis, or psoriatic arthritis), inflammatory brain disease (including post-stroke, encephalitis), atherosclerosis, traumatic injury, infection, and/or shock. In an embodiment, the inflammatory disease is Chronic Obstructive Pulmonary Disease (COPD), such as emphysema, chronic bronchitis, or refractory (non-reversible) asthma.

In embodiments, the inflammatory disease is enteric fistula, chronic radiation damage (which causes inflammatory tissue defects such as radiation cystitis or radiation enteritis), duodenal ulcers, or a chronic inflammatory disease of the central nervous system, such as post stroke neuro-inflammation, schizophrenia, autism, addiction, chronic traumatic encephalopathy, or vaccine induced neuro-toxicity.

In an embodiment, the autoimmune disease is Myasthenia gravis (MG), Hashimoto's thyroiditis, vasculitis, Graves' disease, psoriasis, Chronic inflammatory demyelinating polyneuropathy (CIDP), Guillain barré syndrome, diabetes mellitus type 1, lupus, multiple sclerosis, rheumatoid arthritis, Addison's disease, Sjogren's syndrome, celiac disease, myositis, ankylosing spondylitis, or scleroderma.

In an embodiment, the fibrosis is pulmonary fibrosis (replacement fibrosis, focal fibrosis, diffuse parenchymal lung disease, cystic fibrosis, fibrothorax, idiopathic pulmonary fibrosis, and radiation-induced lung injury), liver fibrosis (bridging fibrosis, cirrhosis, senescence of hepatic stellate cells), kidney fibrosis (CYR61 induction of cellular senescence), glial scars, heart fibrosis (interstitial fibrosis, replacement fibrosis, and myocardial fibrosis) arterial stiffness, athrofibrosis, chronic kidney disease, Crohn's disease, Dupuytren's contracture, keloid, mediastinal fibrosis, myelofibrosis, Peyronie's disease, nephrogenic systemic fibrosis, progressive massive fibrosis, retroperitoneal fibrosis, scleroderma/systemic sclerosis, and adhesive capsulitis.

In an embodiment, the infectious disease is caused by bacteria, virus, or fungus. In an embodiment, the infectious disease is caused by a virus. In an embodiment, the virus is a rhinovirus, coronavirus, influenza, or respiratory syncytial virus. In an embodiment, the coronavirus is severe acute respiratory syndrome coronavirus 2 (SARS-CoV-2).

In an embodiment, the infectious disease causes or is capable of causing cytokine storm in the subject. In an embodiment, the inflammatory disease causes or is capable of causing cytokine storm in the subject.

In embodiments, the therapeutic molecule treats the disease. In embodiments, the therapeutic molecule is a cytokine, a therapeutic antibody, a therapeutic fusion protein, an RNA, a peptide, or a polypeptide. In embodiments, the cytokine is an anti-inflammatory cytokine. In embodiments, the cytokine is selected from interleukin (IL)-1 receptor antagonist, IL-4, IL-6, IL-10, IL-11, IL-13, IFN-alpha, and transforming growth factor-beta. In embodiments, the therapeutic molecule is selected from abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), and muromonab (Orthoclone OKT3).

In embodiments, the therapeutic molecule improves treatment of the disease. For example, the therapeutic molecule may be a receptor that facilitates uptake of a therapeutic agent by a cell that expresses the therapeutic molecule. In another example, the therapeutic molecule may be an antigen recognized by a therapeutic agent. In another example, the therapeutic molecule may be an enzyme that is used by a cell to produce a therapeutic agent (e.g., a steroid). In embodiments, the therapeutic agent is an agent that treats the disease.

In embodiments, the method further includes administering a therapeutic agent to the subject. In embodiments, the therapeutic agent is in the same composition as the oncolytic virus and immune cell. In embodiments, the therapeutic agent is administered separately from the oncolytic virus and immune cell. In embodiments, the therapeutic agent is an agent that treats the disease. In embodiments, the therapeutic agent is selected from abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), and muromonab (Orthoclone OKT3).

In embodiments, the oncolytic virus is administered in a therapeutically effective amount, e.g., at an amount sufficient to treat the disease. In embodiments, the immune cell is administered in a therapeutically effective amount, e.g., at an amount sufficient to treat the disease. In embodiments, the oncolytic virus and immune cell are in the same (a single) composition.

The amount of poxvirus administered to an average-sized adult can be, for example, 1×10² to 1×10¹⁰ plaque-forming units, 1×10³ to 1×10⁸ plaque-forming units, 1×10⁴ to 1×10⁶ plaque-forming units, or any value or sub range there between. As a specific example, about 2.5×10⁵ plaque-forming units can be used.

PCT Patent Publication No. WO 2020/247385 is hereby incorporated by reference in its entirety for all that is taught therein.

### IV. Compositions

In one aspect, provided herein is a composition comprising an oncolytic virus (e.g. a poxvirus, e.g., smallpox, for example a smallpox vaccine) and an immune cell. In embodiments, the oncolytic virus comprises a recombinant polynucleotide, wherein said recombinant polynucleotide encodes a therapeutic molecule.

In some embodiments, the compositions disclosed herein comprise a pharmaceutically acceptable carrier. As used herein, the term “pharmaceutically acceptable carrier” refers to solvents, diluents, preservatives, dispersion or suspension aids, isotonic agents, thickening or emulsifying agents, solid binders, and lubricants, appropriate for the particular dosage form. The skilled artisan is aware of a variety of different carriers that may be used in formulating pharmaceutical compositions and knows techniques for the preparation thereof (See Remington's Pharmaceutical Sciences Ed. by Gennaro, Mack Publishing, Easton, Pa., 1995; which is incorporated herein in its entirety by reference). The pharmaceutically acceptable carriers may include, but are not limited to Ringer's solution, isotonic saline, starches, potato starch, sugars, glucose, powdered tragacant, malt, gelatin, talc, cellulose and its derivatives, ethyl cellulose, sodium carboxymethyl cellulose, cellulose acetate excipients, cocoa butter, suppository waxes, agar, alginic acid, oils, cottonseed oil, peanut oil, safflower oil, sesame oil, olive oil, soybean oil, corn oil, glycols, propylene glycol, esters, ethyl laureate, ethyl oleate, buffering agents, aluminum hydroxide, magnesium hydroxide, phosphate buffer solutions, pyrogen-free water, ethyl alcohol, other non-toxic compatible lubricants, sodium lauryl sulfate, magnesium stearate, coloring agents, releasing agents, coating agents, sweetening, flavoring and perfuming agents. Pharmaceutically acceptable carriers may also include preservatives and antioxidants. One or more of the above-mentioned materials can be specifically excluded from the compositions and methods of some embodiments.

The effective dosage of each of the treatment modalities disclosed herein may vary depending on various factors, including but not limited to the particular treatment, compound or pharmaceutical composition employed, the mode of administration, the condition being treated, and/or the severity of the condition being treated. Thus, the dosage regimen of the combination of the invention is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

Methods of preparing pharmaceutical compositions comprising the relevant treatments disclosed herein are known in the art and will be apparent from the art, from known standard references, such as Remington's Pharmaceutical Sciences, Mack Publishing Company, Easton, Pa., 18th edition (1990), which is incorporated herein by reference in its entirety.

It should be understood that the embodiments described herein are not limited to vaccinations or vaccinating per se, but also relate to generating an immune response or reaction to an antigen associated with a disease. While the words “vaccine,” “vaccination,” or other like terms are used for convenience, it should be understood that such embodiments also relate to immune compositions, immunogenic compositions, immune response generation, immunization, etc., where absolute prophylactic immunity is not required or generated. For example, the embodiments referring to vaccination also can relate to generating or to assisting in creating an immunogenic or immune response against an antigen, regardless of whether that response results in absolute eradication or immunization against the disease to be treated.

U.S. Pat. No. 10,105,436, which is incorporated herein by reference in its entirety, describes methods of administering, making, storing, and using compositions comprising poxvirus and cells that can be used in the methods and compositions described herein.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Preparation of Patient-Derived CAR T Cells

Patient derived cells were expanded and cultured in the presence of IL7 and IL15 as reported previously (Quintarelli et al., Oncoimmunology. 2018; 7 (6): e1433518, incorporated herein by reference in its entirety); the same procedure is used in phase I/II clinical studies presently ongoing (NCT03373097 and NCT03373071).

Briefly, peripheral blood mononuclear cells (PBMC) were isolated from buffy coats obtained from healthy donors, using Lymphocytes separation medium. T lymphocytes were activated with immobilized OKT3 (1 μg/ml) and anti-CD28 (1 μg/ml) monoclonal antibody (mAb) in the presence of recombinant human interleukin-2 (IL2, 100 U/ml), or with a combination of recombinant human interleukin-7 (IL7, 10 ng/ml) and 15 (IL15, 5 ng/ml). Activated T cells were transduced on day 3 in plates pre-coated with recombinant human RetroNectin using supernatant and the specific above-described cytokines. On day 5 after transduction, T cells were expanded in medium containing 45% RPMI1640 and 45% Click's medium (Sigma-Aldrich, Co.; USA) supplemented with 10% FBS and 2 mM Glutamax, and replenished twice a week.

### Example 2: Infection of CAR T Cells with Vaccinia Virus

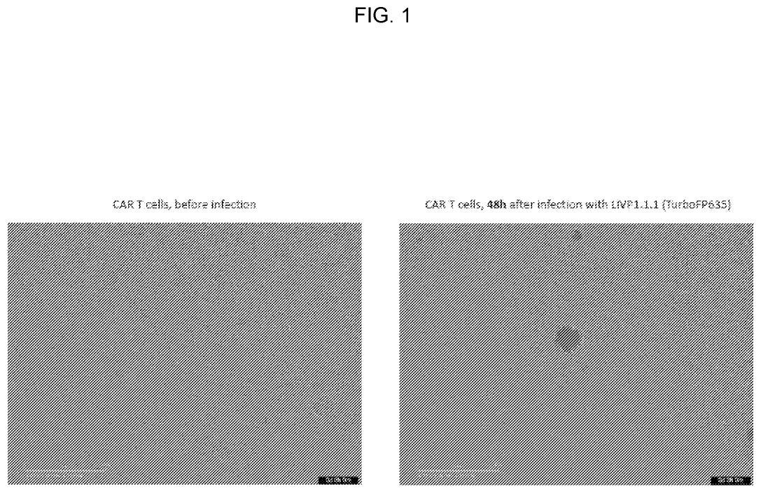

T cells used in the studies shown in FIGS. 1-9B were infected in each experiment in the presence of culture medium. An example infection included infecting approximately 5×10⁵ cells/ml with 2.5×10⁶ VV PFU (MOI 5) for 2 to 3 days at 37° C. Infected cells were washed three times prior to any co-culture experiments.

Vaccinia virus can infect both polyclonally activated T cells and chimeric antigen receptor (CAR) T cells. In addition, vaccinia virus can replicate within T cells to create new viral progenies. FIG. 1 shows fluorescent micrographs of CAR T cells before (left panel) and 48 hours (h) after infection with vaccinia virus (LIVP1.1.1) (right panel).

### Example 3: Cell Assays with CAR T Cells Infected with Vaccinia Virus

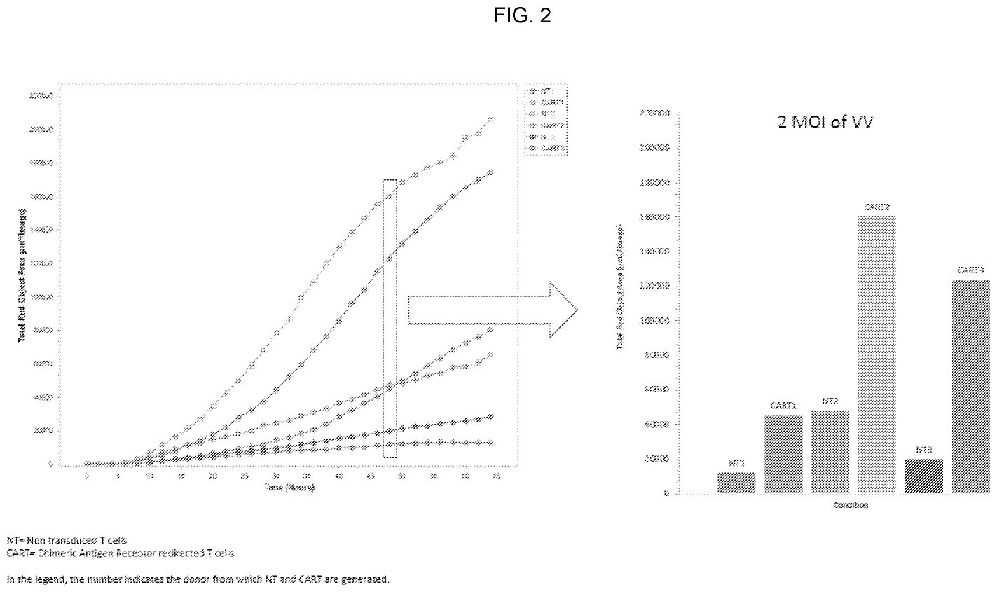

FIG. 2. Shows two plots that indicate that CAR T cells are more permissive to infection by vaccinia virus than non-transduced (NT) T cells. The left plot shows the total virus infected area (μm²/image) over time (hours) for three sets of CAR vs. NT cells generated from three patients. The right plot shows the total virus infected area (μm²/image) at the time course end point (65 hours) for each condition (patients 1, 2, and 3 CART and NT samples).

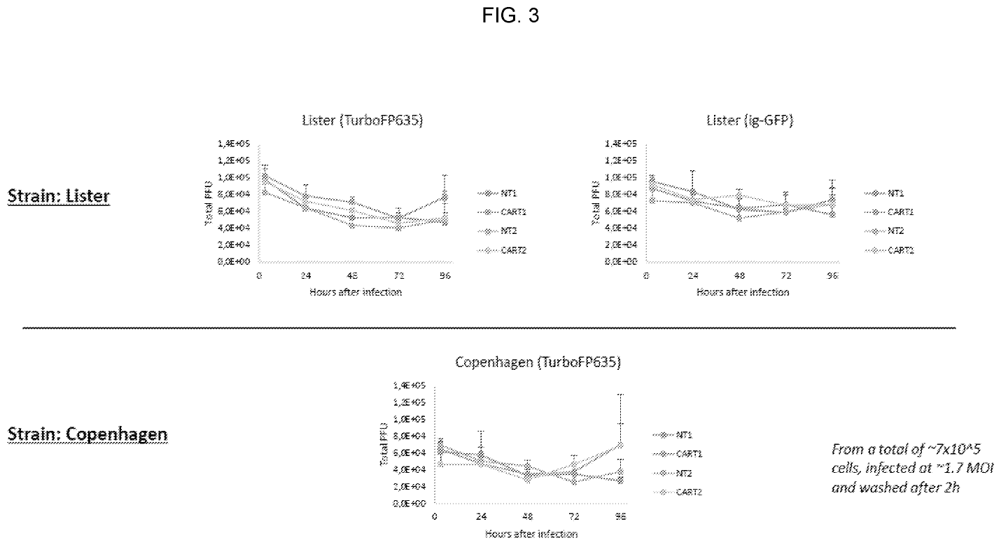

FIG. 3. Shows that there are low levels of virus spontaneously released over 96 hours by previously infected CAR T/NT cells for two strains of vaccinia virus. In the top two plots, the Lister strain (variants TurboFP635 and ig-GFP) was used, and in the bottom plot, the Copenhagen strain (TurboFP635 variant) was used. In each case, a total of ˜7×10^{cells} were infected at ˜1.7 MOI and washed after 2 hours. The total plaque forming units (PFU) were counted and plotted at 24, 48, 72, and 96 hours.

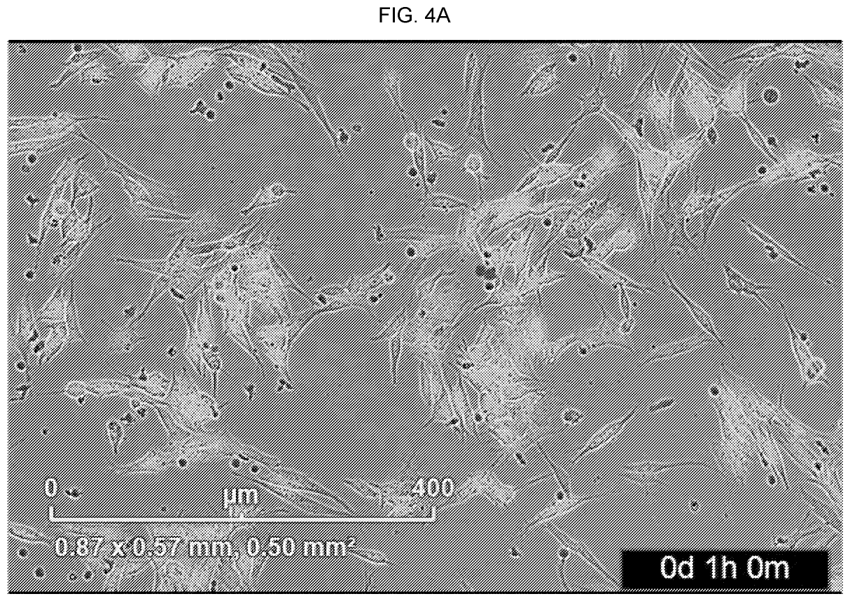

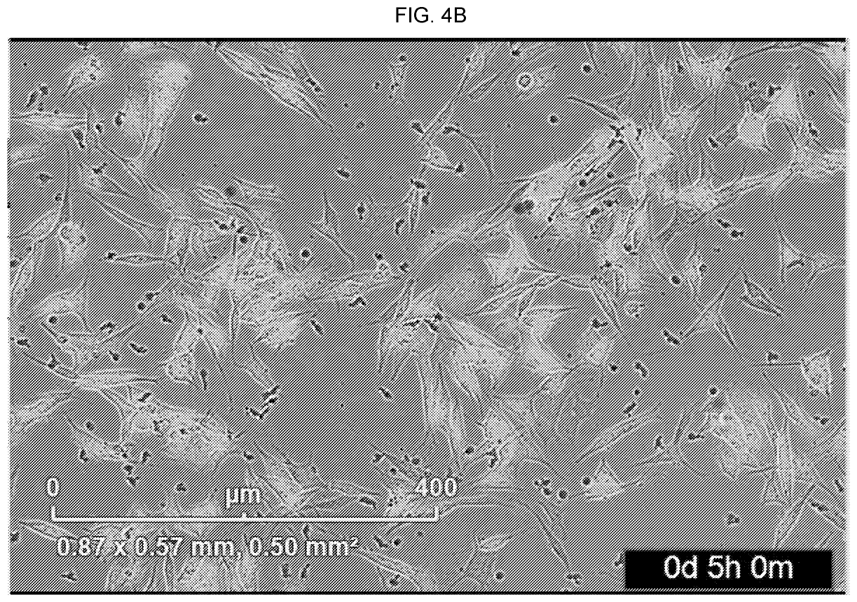

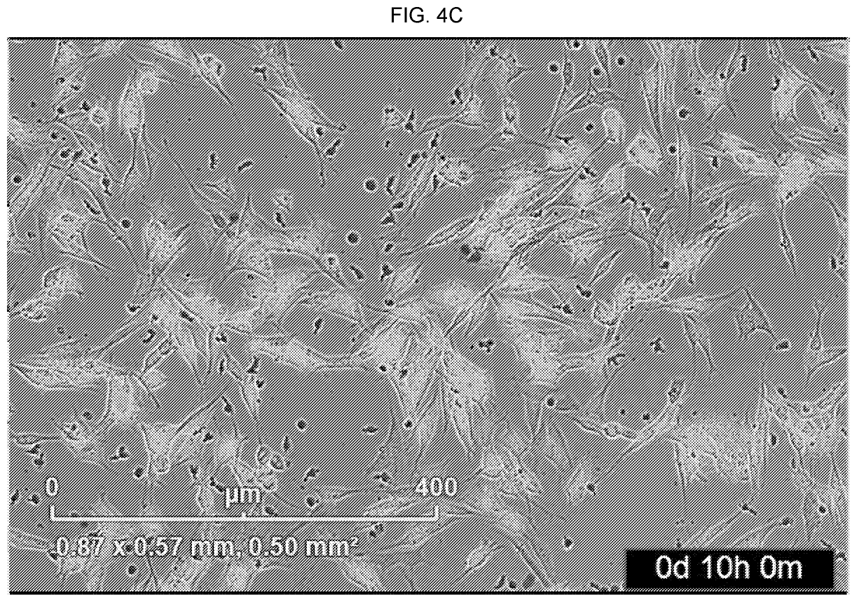

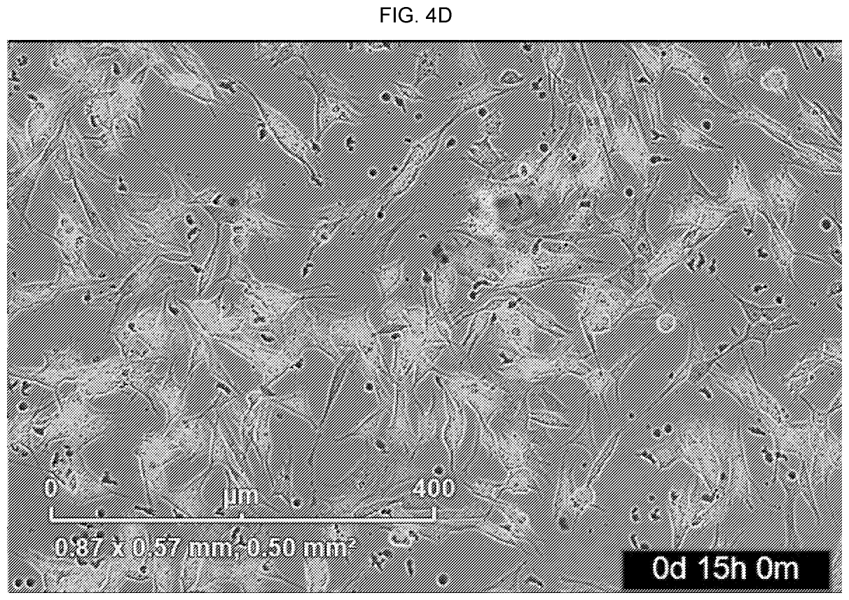

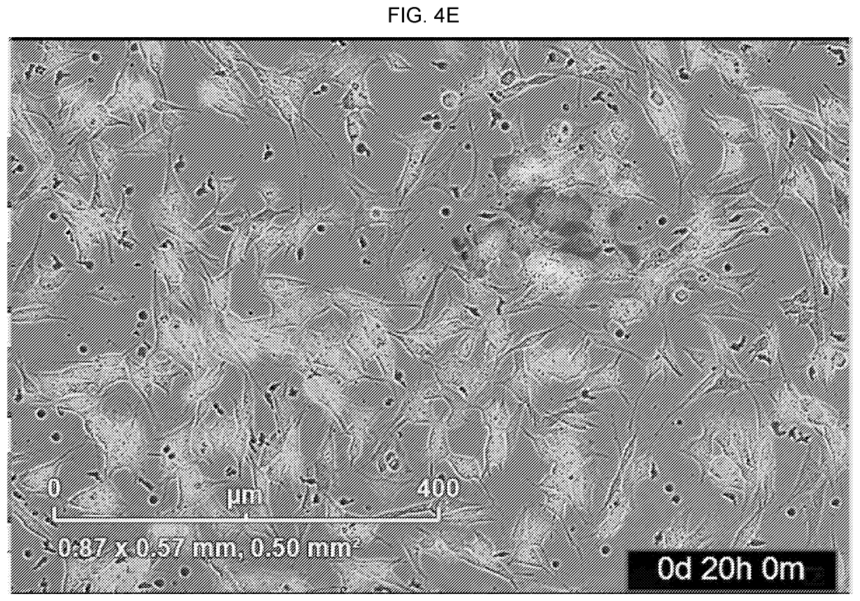

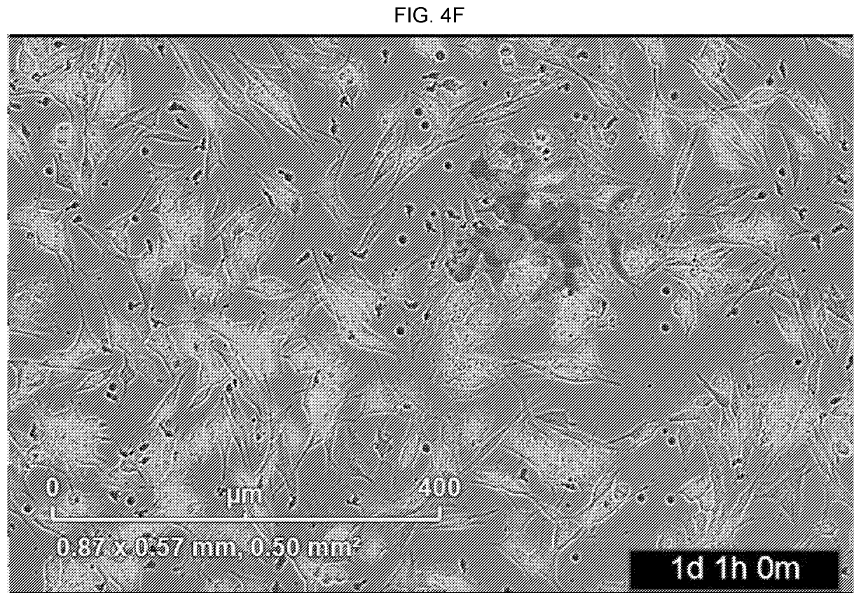

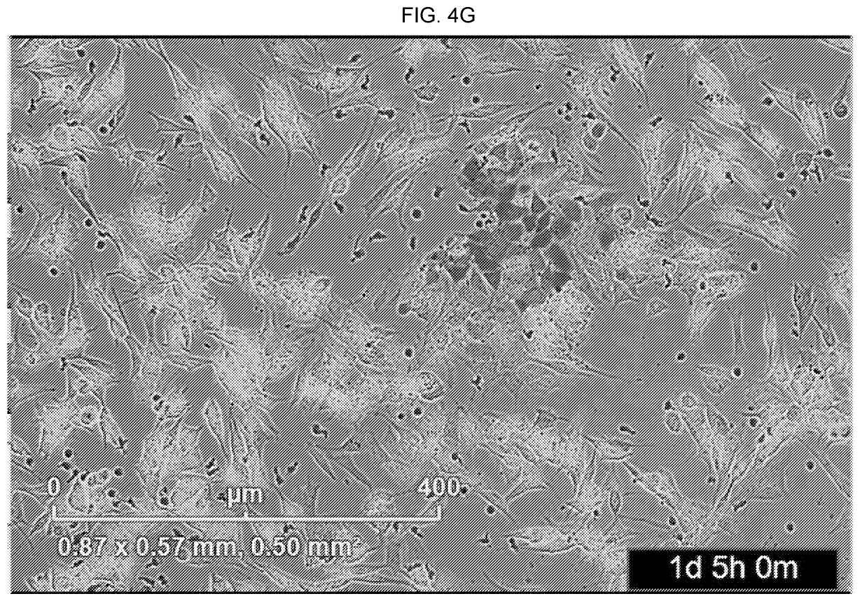

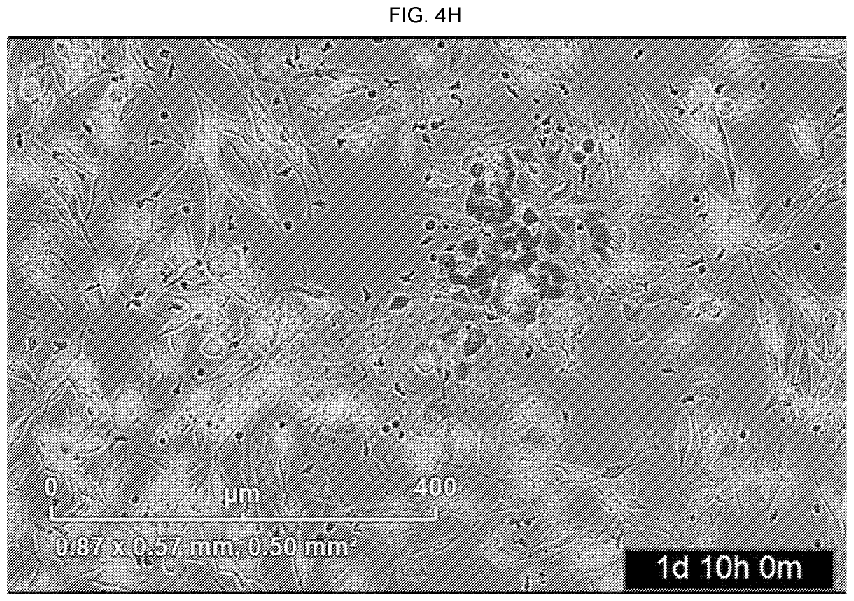

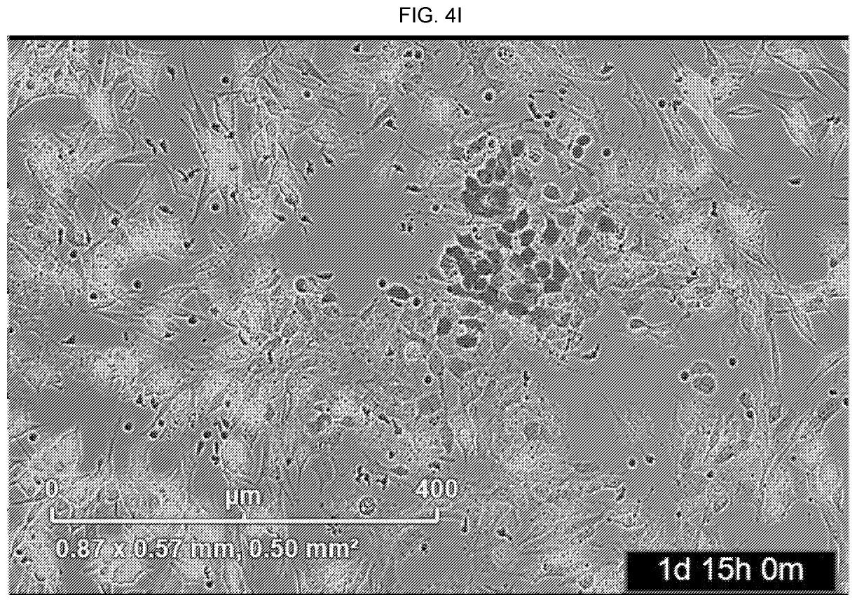

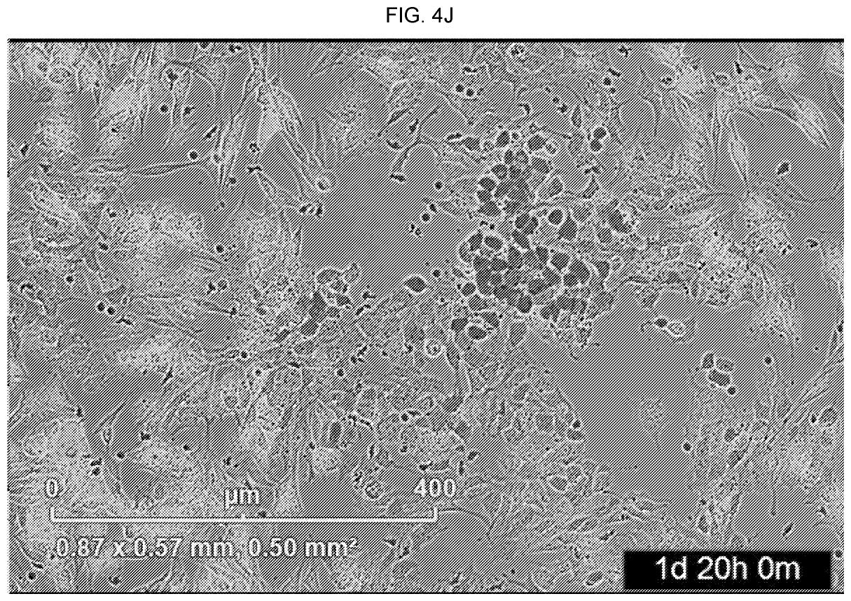

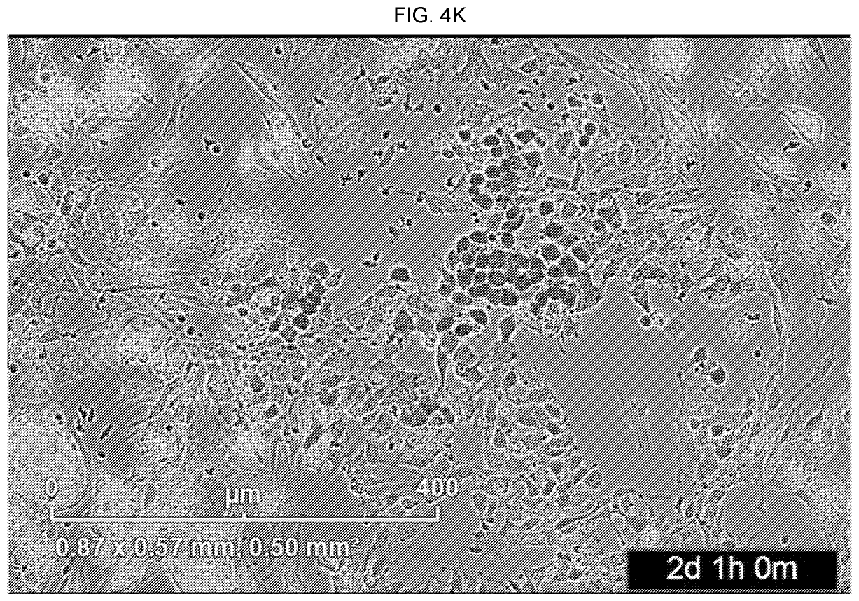

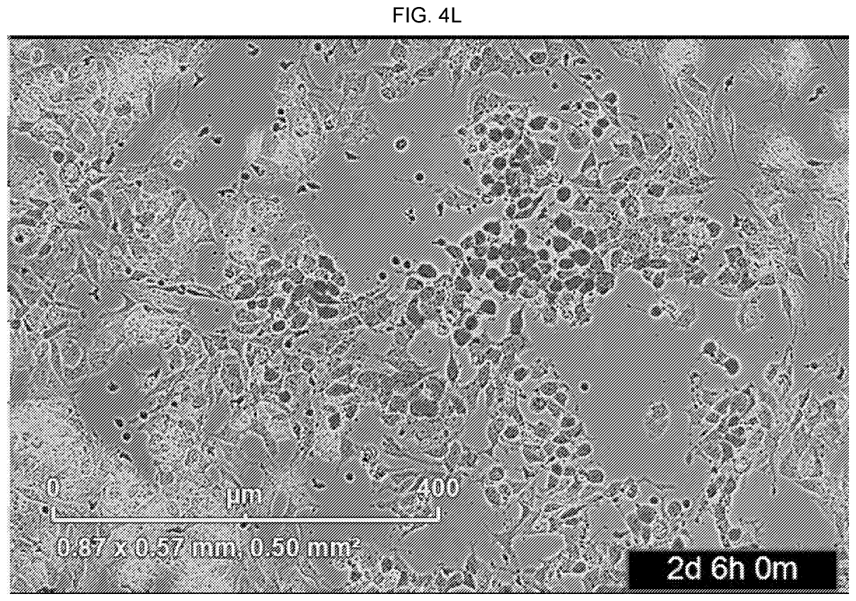

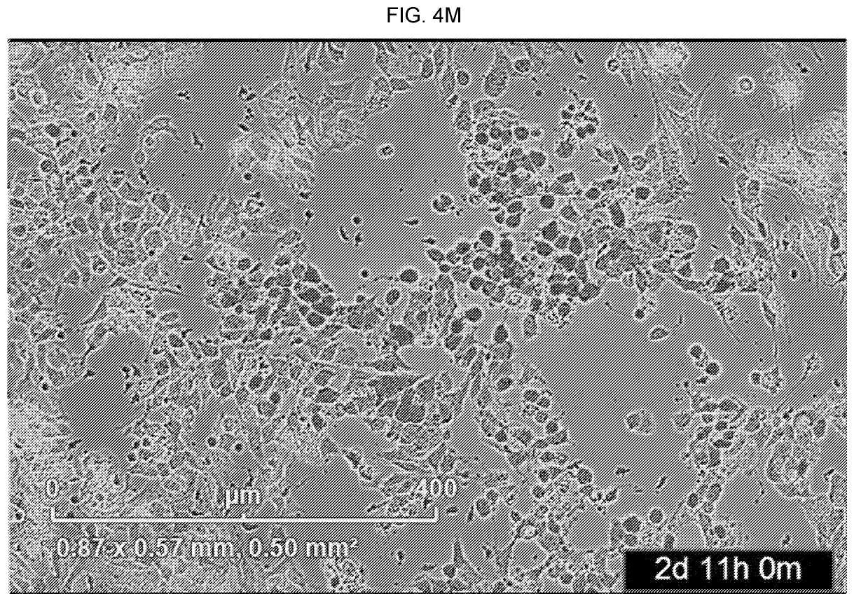

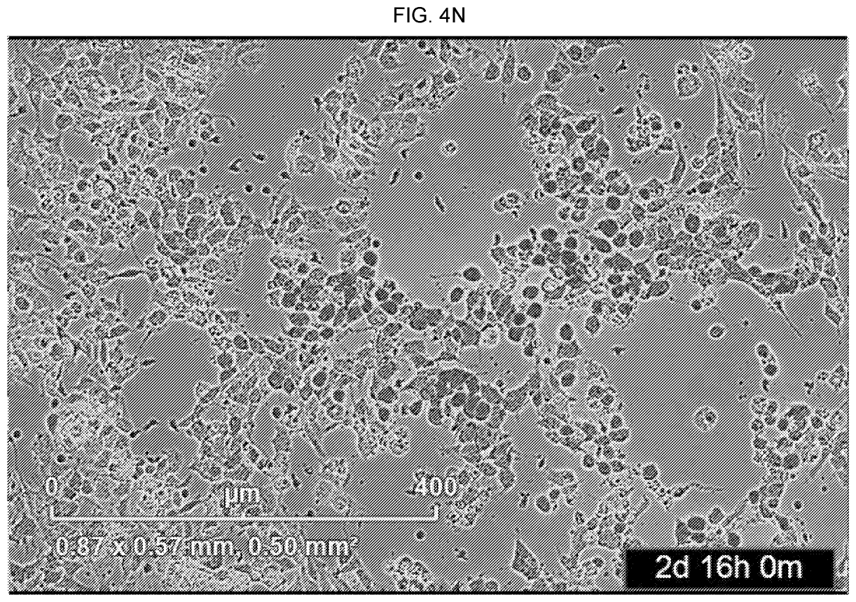

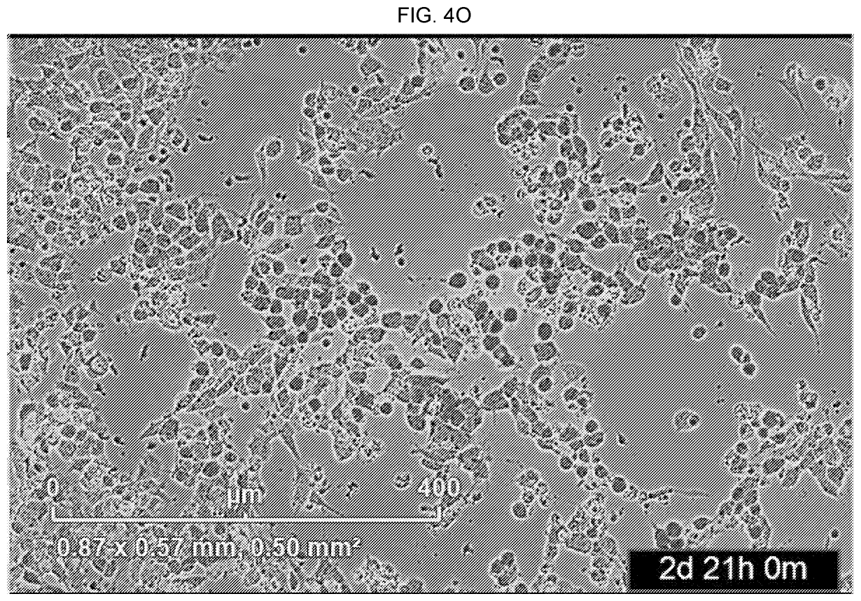

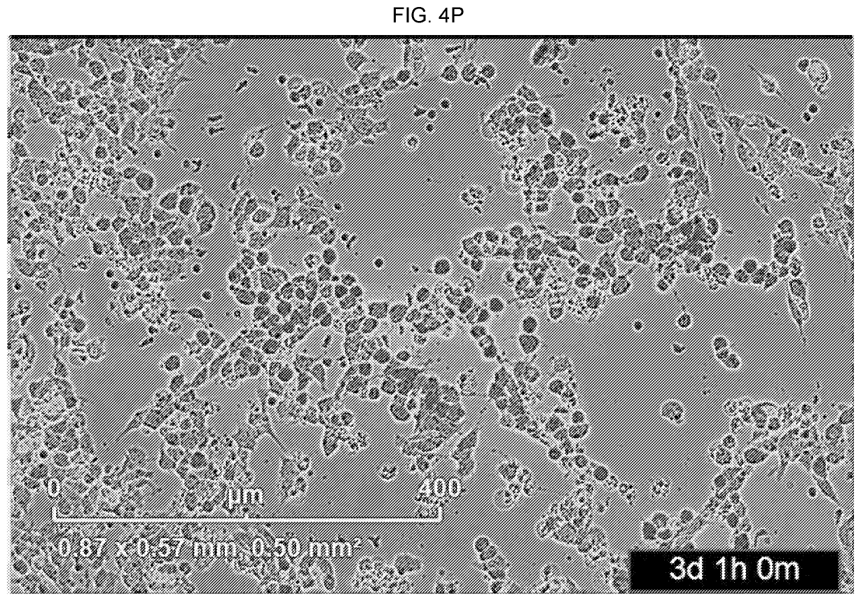

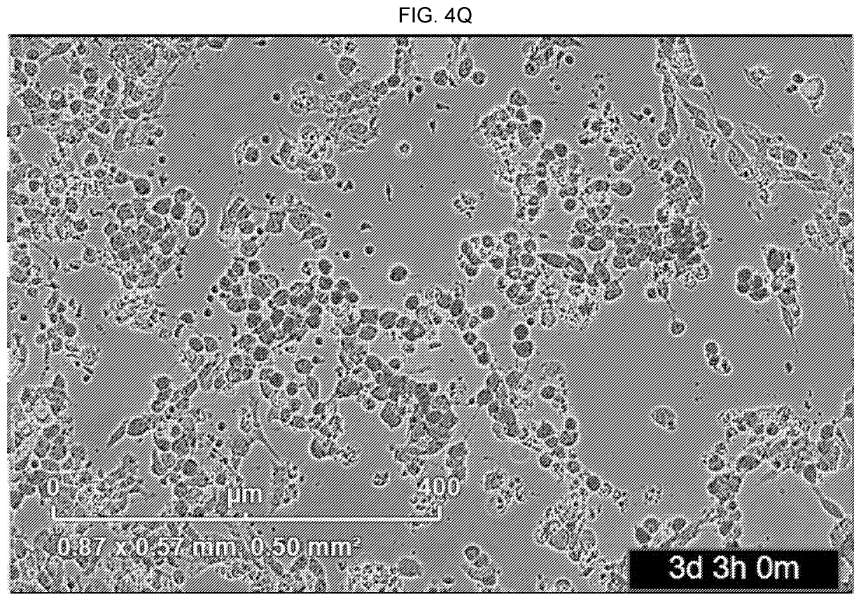

FIG. 4. Shows that vaccinia virus released by previously infected T cells can infect tumor cells in vitro. Also it was found that vaccinia infected T cells remain functional, and the vaccinia virus is released from the infected T cells only after engagement of CAR receptors or exogenous T cell receptor to the target tumor cell. The vaccinia infected tumor cells are lysed and release new vaccinia particles.

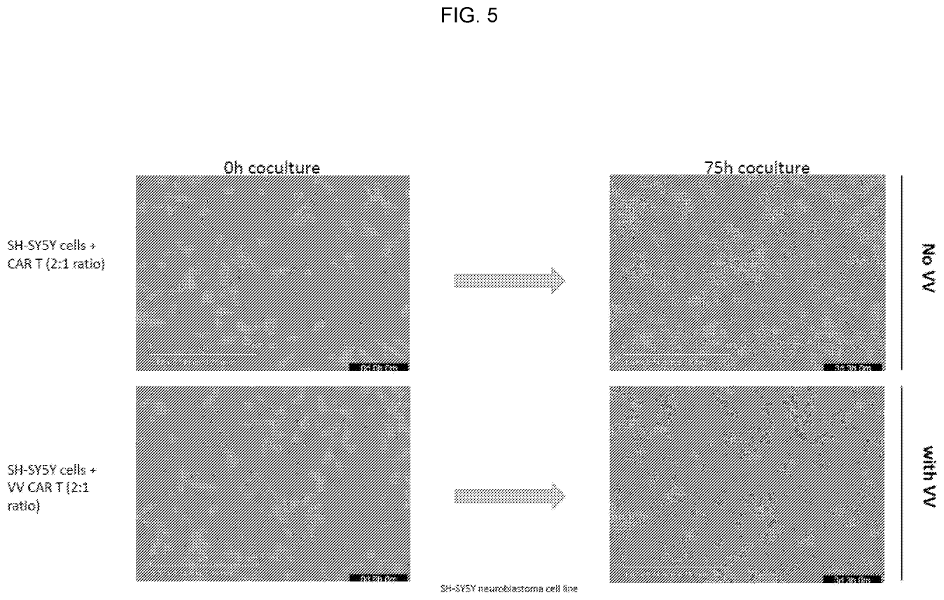

FIG. 5. Shows that CAR T cells infected with vaccinia virus eradicate more SH-SY5Y neuroblastoma cells over time than CAR T cells alone. SH-SY5Y cells were co-cultured 2:1 with CAR T cells (top row) or CAR T cells previously infected with vaccinia virus (bottom row). There was a significant enhancement of tumor cell eradication over 75 hours for the tumor cells co-cultured with the vaccinia virus-infected CAR T cells as compared to the tumor cells co-cultured with CAR T cells alone. These data represent in vitro co-culture that is closer to in vivo conditions, with more tumor cells than T cells in the co-culture.

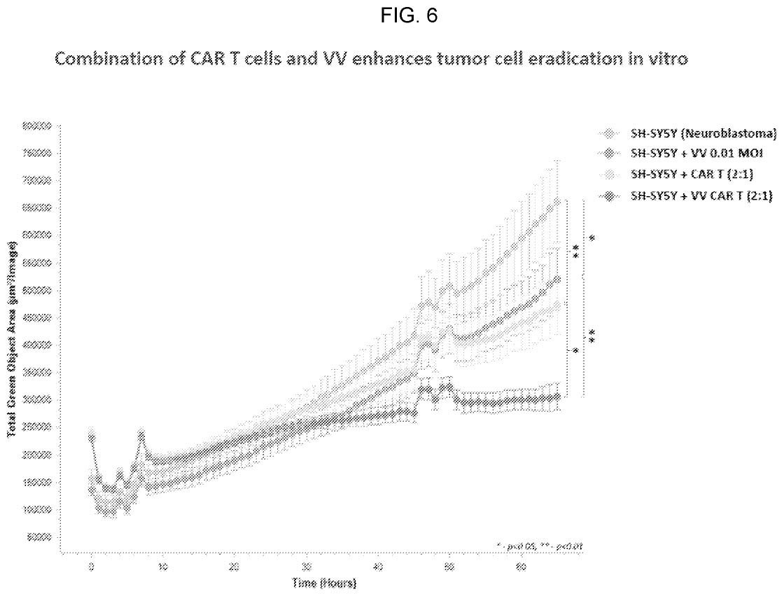

FIG. 6. Shows a plot of the of the total green object area (viable cells) over time (hours), indicating the amount of intact tumor cells in culture, either alone, + vaccinia virus at 0.01 MOI, + CAR T cells (2:1 co-culture), or with vaccinia virus-infected CAR T cells.

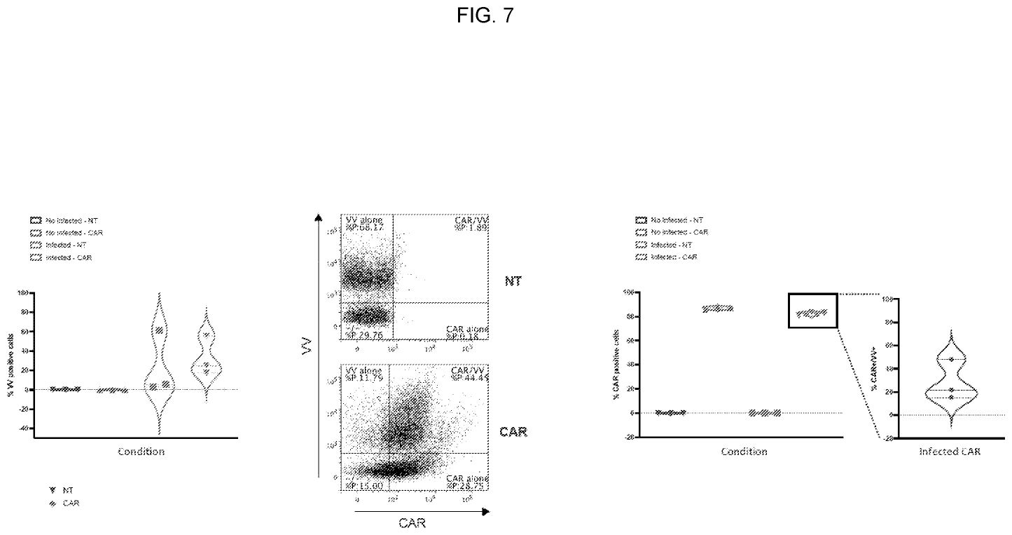

FIG. 7. Shows the number of vaccinia-infected T cells under the indicated conditions. The two plots on the left represent FACS analysis data (%) of Vaccinia infected polyclonally-activated T cells and the two plots on the right show FACS analysis of modulation of CAR expression after Vaccinia virus infection. Right plot shows CAR expression under each condition, as % CAR positive cells. Vaccinia virus infection is more homogenous in CAR redirected T cells compared to non-gene modified T cells, and vaccinia infection does not negatively affect the level of CAR T cell expression. No infected—NT: Uninfected T cells; No infected—CAR: Uninfected CAR T cells; Infected—NT: Vaccinia-infected T cells; Infected—CAR: Vaccinia-infected CAR T cells.

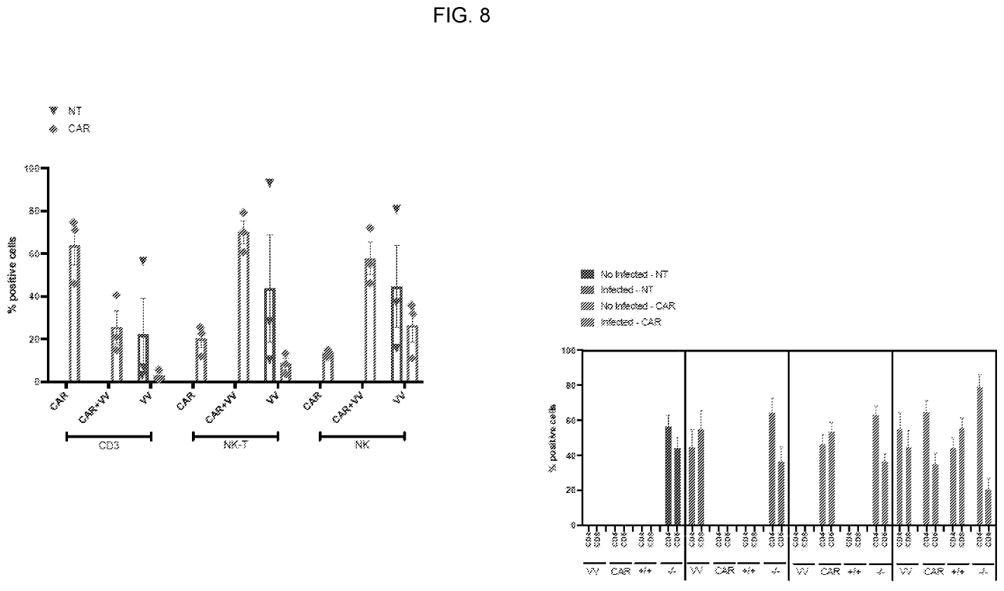

FIG. 8. The left plot shows that vaccinia virus infects T cells, NK cells, and NK-T cells. FACS analysis of subpopulation of cells present in patient derived CAR gene-modified T cells. The left panel shows percentages of T, NK, NK-T populations in the non-gene modified (NT-non-transduced) T cells, and gene-modified CAR after Vaccinia infection. On the right, FACS analysis of CD8 and CD4 compartments in CD3+ population with or without Vaccinia infection in both NT and CAR+ patient derived lymphocytes. CAR detection: anti CAR idiotype. T cell populations include: CD45/CD3, NK-T: CD45/CD3/CD56, NK: CD45/CD56+/CD3−. The right plot shows that vaccinia virus infects CD4 and CD8 subpopulations of T cells. The percentage of infection in CD8 cells is greater than in CD4 cells in this example. The right panel shows % positive cells vs., from the left to the right: VV+: only VV positive cells, CAR+: only CAR positive cells, +/+: cells positive for both VV and CAR, −/−: cells that are double negative for VV and CAR.

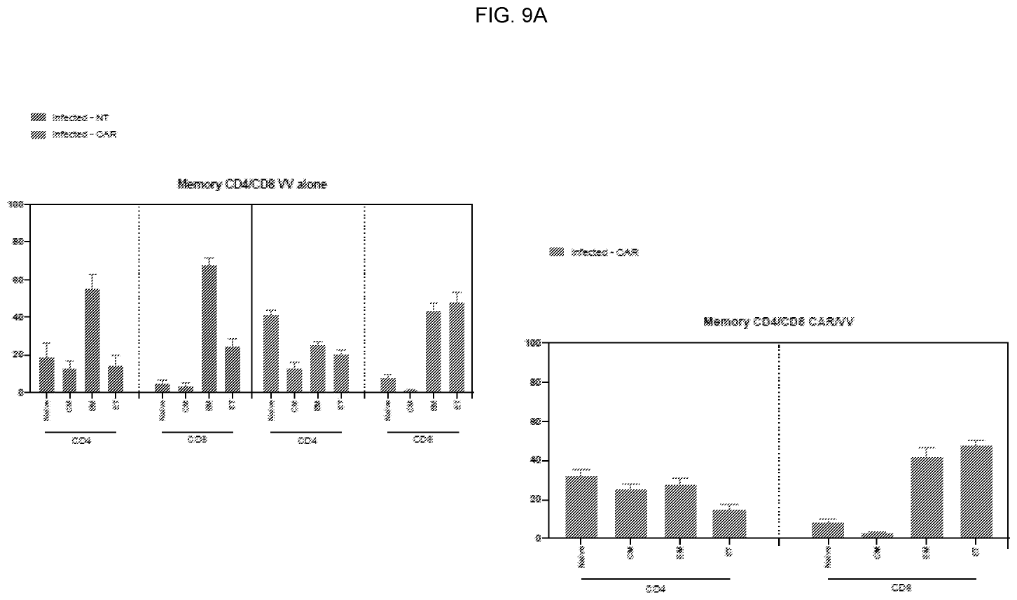

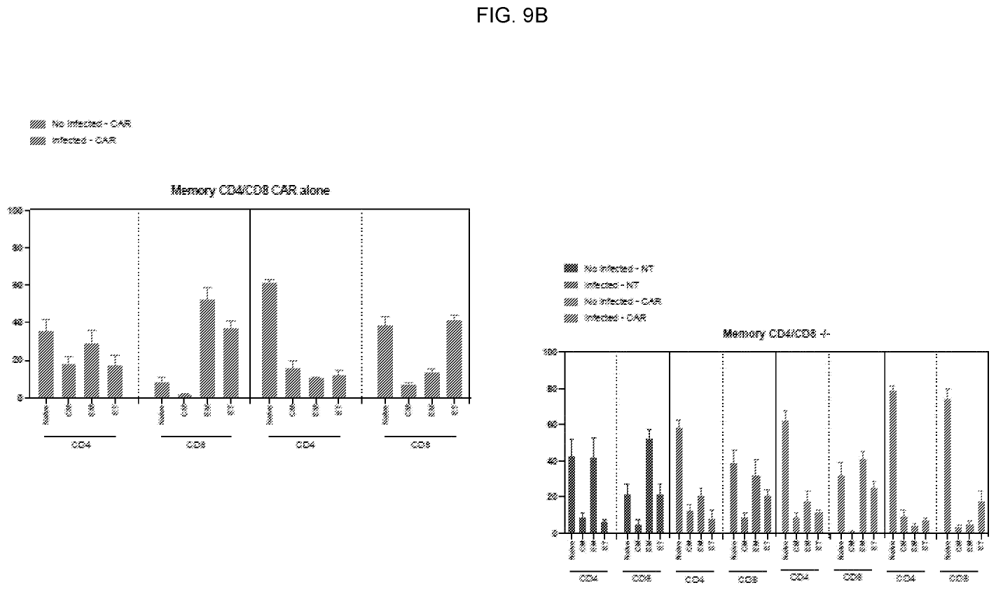

FIG. 9A. The vaccinia infection induces a maturation of T cells mainly in the CD8 compartment, with a minor amount of naïve cells and major amount of effector cells. FIG. 9B. The vaccinia infection induces a maturation of T cells mainly in the CD8 compartment, with a minor amount of naïve cells and major amount of effector cells. FIGS. 9A and 9B show that after VV infection in the CD8 compartment the amount of naïve and memory T cell subpopulations is reduced in comparison to non-Vaccinia infected cells.

### Example 4: Administration of CAR T Cells Infected with Vaccinia Virus

T cells are removed from a patient's blood via apheresis or leukapheresis. The T cells are optionally genetically engineered to express a chimeric antigen receptor of interest. The T cells may be expanded in vitro.

T cells are infected with vaccinia virus at a MOI of about 2. The infected T cells are administered to the patient via intravenous injection.

What is claimed is:

1. A composition comprising an oncolytic virus and an immune cell, wherein the immune cell is infected by the oncolytic virus.

2. The composition of claim 1, wherein the immune cell is a lymphocyte.

3. The composition of claim 2, wherein the lymphocyte is a T-cell, a B-cell, an NK cell, an NK-T cell, or a myeloid cell including lymphoid dendritic cell.

4. The composition of any one of claims 1 to 3, wherein the immune cell is derived from a subject to be treated with the composition.

5. The composition of any one of claims 1 to 3, wherein the immune cell is allogeneic to a subject to be treated with the composition.

6. The composition of any one of claims 1 to 5, wherein the oncolytic virus is a poxvirus.

7. The composition of any one of claims 1 to 6, wherein the immune cell is genetically modified.

8. The composition of claim 7, wherein the immune cell is genetically modified to target a target cell.

9. The composition of claim 8, wherein the target cell is a cancer cell, a fibroblast, a pathogen, or a pathogen-infected cell.

10. The composition of any one of claims 1 to 9, wherein the virus does not lyse the immune cell for at least 5 days after infection.

11. A composition comprising a poxvirus and a T cell or NK cell expressing a chimeric antigen receptor (CAR) (CAR-T cell or CAR-NK cell).

12. A composition comprising a poxvirus and a T cell expressing an exogenous T cell receptor (TCR).

13. The composition of claim 11 or 12, wherein the T cell is derived from a lymphocyte obtained from a subject to be treated with the composition.

14. The composition of claim 11 or 12, wherein the T cell is derived from a lymphocyte obtained from an unrelated donor.

15. The composition of claim 13 or 14, wherein the lymphocyte is a T-cell, NK cell or NK-T cell.

16. The composition of claim 13 or 14, wherein the lymphocyte is a T-cell or NK cell.

17. The composition of any one of claims 11 to 16, wherein the CAR targets an antigen associated with a disease.

18. The composition of claim 17, wherein the disease is cancer.

19. The composition of claim 17, wherein the disease is an infectious disease.

20. The composition of claim 17, wherein the disease is fibrosis.

21. The composition of any one of claims 1 to 20, wherein the poxvirus is a vaccinia virus.

22. The composition of claim 21, wherein the vaccinia virus is selected from Dryvax, ACAM1000, ACAM2000, Lister, EM63, LIVP, Tian Tan, Copenhagen, Western Reserve, Modified Vaccinia Ankara (MVA), New York City Board of Health, Dairen, Ikeda, LC16M8, Western Reserve Copenhagen, Tashkent, Tian Tan, Wyeth, IHD-J, and IHD-W, Brighton, Dairen I and Connaught strains.

23. The composition of claim 22, wherein the vaccinia virus is a Lister strain.

24. The composition of claim 22, wherein the vaccinia virus is a Copenhagen strain.

25. The composition of any one of claims 1 to 24, wherein the poxvirus is oncolytic.

26. The composition of any one of claims 11 to 25, wherein the CAR targets CD5, CD7, CD19, CD20, CD22, CD30, CD33, CD44v6, CD123, CD138, CD171, B7-H3, BCMA, CEA, CSPG4, EGFR, EGFRvIII, EphA2, FAP, FLT3, GD2, Glypican 3, Igκ, Igλ, IL13, Her2, Her3, LeY, Mesothelin, PD-L1, PSMA, ROR1, SLAMF7.

27. The composition of any one of claims 11 to 25, wherein the exogenous TCR targets a tumour associated antigen.

28. The composition of any one of claims 11 to 25, wherein the exogenous TCR targets a fibrosis associated antigen.

29. The composition of claim 27, wherein the tumour associated antigen is PRAME, WT1, Survivin, MAGE-A3, MART-1, gp-100, p53, or NY-ESO1.

30. A pharmaceutical composition comprising the composition of any one of claims 1 to 29 and a pharmaceutically acceptable excipient.

31. A method for treating a disease in a subject, the method comprising administering to the subject a composition of any one of claims 1 to 30.

32. The method of claim 31, wherein the disease is an infection, autoimmune disease, fibrosis, or inflammatory disease.

33. A method for treating cancer in a subject in need thereof, the method comprising administering to the subject a composition of any one of claims 1 to 30.

34. The method of claim 33, wherein the cancer is selected from high-grade glioma, low-grade glioma, non-Hodgkin lymphoma, Hodgkin lymphoma, B and T cell acute lymphoblastic leukemia, mantle cell lymphoma, multiple myeloma, acute myeloid leukemia, chronic lymphoblastic leukemia, chronic myeloid leukemia, sarcomas, neuroblastoma, retinoblastoma, nephroblastoma, medulloblastoma, germinal tumors, desmoid tumors, juvenile myelomonocytic leukemia, lung cancer, breast cancer, colon and rectum cancer, stomach cancer, pancreatic cancer, kidney and bladder cancer, head and neck cancer, melanoma, uterus and prostate cancer.

35. The method of claim 34, wherein the cancer is glioblastoma.

36. The method of any one of claims 31 to 35, wherein the composition is administered to the subject by intravenous, intraperitoneal, intrathecal, intra-cerebro-ventricular, intrapleural, intra-parencymal, intraventricular, intraarticular, or intraocular injection.

37. The method of any one of claims 31 to 35, wherein the composition is administered directly to a region affected by the disease.

38. The method of any one of claims 31 to 37, wherein the composition is administered by MRI-guided delivery.

39. The method of any one of claims 31 to 38, wherein the subject is a human.

40. The method of any one of claims 31 to 39, wherein the immune cell is derived from the subject.

41. The method of any one of claims 31 to 39, wherein the immune cell is allogeneic to the subject.

42. The method of any one of claims 31 to 41, wherein the infected immune cell releases the poxvirus upon binding to a target cell.

43. A method for making a composition comprising a poxvirus and lymphocytes, the method comprising:
(a) obtaining leukocytes from a subject; and
(b) contacting the leukocytes with a poxvirus to form poxvirus-infected leukocytes.

44. The method of claim 43, wherein the leukocytes are isolated prior to step b).

45. The method of claim 43 or 44, wherein the leukocytes are isolated by leukapheresis, peripheral blood or bone marrow.

46. The method of any one of claims 43 to 45, wherein the leukocytes comprise α/β-T cells, γ/δ-T cells, NK-T cells, NK cells.

47. The method of claim 41, wherein the T cells are further polarized.

48. The method of claim 41, wherein the T cells are CD4 or CD8 or CD45RA+, naïve T cells, memory T cells, stem cell-like memory T cells.

49. The method of any one of claims 43 to 48, wherein the T cells comprise chimeric antigen receptor (CAR) (CAR-T cells) and exogenous TCRs.

50. The method of any one of claims 43 to 49, wherein the poxvirus is a vaccinia virus.

51. The method of claim 50, wherein the vaccinia virus is selected from Dryvax, ACAM1000, ACAM2000, Lister, EM63, LIVP, Tian Tan, Copenhagen, Western Reserve, Modified Vaccinia Ankara (MVA), New York City Board of Health, Dairen, Ikeda, LC16M8, Western Reserve Copenhagen, Tashkent, Tian Tan, Wyeth, IHD-J, and IHD-W, Brighton, Dairen I and Connaught strains.

52. The method of claim 50, wherein the vaccinia virus is a Lister strain.

53. The method of claim 50, wherein the vaccinia virus is a Copenhagen strain.

54. The method of any one of claims 43 to 53, wherein the lymphocytes are infected with the poxvirus at a titer >0.5 MOI.

55. The method of claim 54, wherein the lymphocytes are infected with the poxvirus at a titer >1 MOI.

56. The method of any one of claims 43 to 55, wherein the lymphocytes are incubated with the virus for about 1 day to about 5 days.

57. The method of claim 56, wherein the lymphocytes are incubated with the virus for about 3 days.

58. The method of any one of claims 43 to 57, wherein the CAR or the exogenous TCR targets an antigen associated with a disease.

59. The method of 58, wherein the CAR targets CD5, CD7, CD19, CD20, CD22, CD30, CD33, CD44v6, CD123, CD138, CD171, B7-H3, BCMA, CEA, CSPG4, EGFR, EGFRvIII, EphA2, FAP, FLT3, GD2, Glypican 3, Igκ, Igλ, IL13, Her2, Her3, LeY, Mesothelin, PD-L1, PSMA, ROR1, SLAMF7.

60. The method of 58, wherein the exogenous TCR targets a tumour associated antigen.

61. The method of claim 60, wherein the tumour associated antigen is PRAME, WT1, Survivin, MAGE-A3, MART-1, gp-100, p53 or NY-ESO1.

62. The method of any one of claims 58 to 61, wherein the disease is a cancer.

63. The method of any one of claims 58 to 61, wherein the disease is an infection, autoimmune disease, fibrosis, or inflammatory disease.

64. The method of any one of claims 43 to 63, wherein the poxvirus does not lyse the lymphocyte.

65. The method of any one of claims 43 to 64, wherein the lymphocytes are derived from a subject to be treated with the composition.

66. The composition of any one of claims 43 to 64, wherein the lymphocytes are allogeneic to a subject to be treated with the composition.
